(19) United States
(12) Patent Application Publication
(10) Pub. No.: US 2004/0002058 A1
(43) Pub. Date: Jan. 1, 2004

(54) Chimeric capsid proteins and uses thereof

(75) Inventor: Larry Cosenza, Birmingham, AL (US)

Correspondence Address: NEEDLE & ROSENBERG, P.C., SUITE 1000, 999 PEACHTREE STREET, ATLANTA, GA, 30309-3915, US

(73) Assignee: UAB Research Foundation

(21) Appl. No.: 10/329,987

(22) Filed: Dec. 26, 2002

Related U.S. Application Data
(63) Continuation-in-part of application No. 10/176,714, filed on Jun. 21, 2002.
(60) Provisional application No. 60/300,044, filed on Jun. 21, 2001.

(51) Int. Cl. C12Q 1/70; G06F 19/00; G01N 33/48; G01N 33/50; C07K 14/005

(52) U.S. Cl. 435/5; 530/350; 702/19

(57) ABSTRACT

The present invention encompasses chimeric capsid proteins, nucleic acids encoding such proteins and capsids containing chimeric capsid proteins. Methods of making the chimeric capsid proteins, the nucleic acids that encode such proteins and capsids that contain chimeric capsid proteins are also encompassed within the scope of the invention. The invention further encompasses the use of the chimeric capsid proteins to produce protein elements and to present the elements for use in structure-function studies, for use as therapeutic factors and for other purposes. Other embodiments of the invention will be apparent to those skilled in the art from consideration of the specification and practice of the invention disclosed herein. It is intended that the specification and examples be considered as exemplary only.

This application claims priority to U.S. Provisional Application Serial No. 60/300,044, filed Jun. 21, 2001, which application is herein incorporated by reference in its entirety.

This invention was made with government support under NASA Grant NAS8-01156. The government may have certain rights in the invention.

### FIELD OF THE INVENTION

This invention relates generally to chimeric phage or viral capsid proteins, capsids made from the chimeric capsid proteins, and uses of both the capsids and capsid proteins. More particularly, the invention relates to chimeric proteins wherein the heterologous portion of the chimeric protein, that corresponding to the non-capsid protein sequences of the chimeric protein, lies on the interior surface of assembled capsids, to capsids formed by the chimeric proteins and uses of both the chimeric proteins and capsids.

### SUMMARY OF THE INVENTION

In accordance with the purpose(s) of this invention, as embodied and broadly described herein, this invention, in one aspect, relates to a chimeric capsid protein which contains a first polypeptide sequence and a second polypeptide sequence. The first polypeptide sequence consists of native capsid protein amino acid sequence. The second polypeptide sequence consists of a heterologous non-capsid amino acid sequence. The second polypeptide sequence comprised in the chimeric capsid protein is displayed on the surface of the chimeric capsid protein which lies on the inner surface of a phage or viral capsid formed from the capsid protein.

In various preferred embodiments of the first aspect of the invention, the first polypeptide sequence is derived from a phage. Suitable phages include, but are not limited to, bacteriophage FR, bacteriophage G4, bacteriophage GA, bacteriophage HK97, bacteriophage HK97 prohead II, bacteriophage MS2, bacteriophage PP7, bacteriophage Qβ and bacteriophage ΦX174. The phage from which the first polypeptide sequence is derived can be an unenveloped phage. Further, an unenveloped phage, as defined herein, can also mean a normally enveloped phage from which the envelope has been removed or for which the envelope has not been allowed to form during assembly of the phage particle. The phage from which the first polypeptide sequence is derived can be an isometric phage.

In various preferred embodiments of the first aspect of the invention, the first polypeptide sequence is derived from a virus. Suitable viruses include, but are not limited to, echovirus 1, hepatitis B virus, alfalfa mosaic virus, bean pod mottle virus, black beetle virus, bluetongue virus, bovine enterovirus, carnation mottle virus, cowpea chlorotic mottle virus, cowpea mosaic virus, coxsackievirus B3, cricket paralysis virus, cucumber mosaic virus, densovirus, desmodium yellow mottle virus, feline panleukopenia virus, flock house virus, foot and mouth disease virus, human rhinovirus 16, human rhinovirus HRV1A, human rhinovirus serotype 2, human rhinovirus serotype 3, human rhinovirus serotype 14, meno encephalomyocarditis virus, nodamura virus, Norwalk virus, nudaurelia capensis ω virus, pariacoto virus, physalis mottle virus, poliovirus type 1, poliovirus type 2, poliovirus type 3, red clover mottle virus, reo virus, rice yellow mottle virus, satellite panicum mosaic virus, satellite tobacco mosaic virus, satellite tobacco necrosis virus, sesbania mosaic virus, southern bean mosaic virus, simian virus 40, murine polyomavirus, Theiler MEV DA, Theiler MEV BeAn, tobacco necrosis virus, tobacco ringspot virus, tomato bushy stunt virus, turnip crinkle virus and turnip yellow mosaic virus. The virus from which the first polypeptide sequence is derived can be an unenveloped virus. Further, an unenveloped virus, as defined herein, can also mean a normally enveloped virus from which the envelope has been removed or for which the envelope has not been allowed to form during assembly of the viral particle.

In various preferred embodiments of the first aspect of the invention, the second polypeptide sequence is derived from a species different from the species from which the first polypeptide is derived. The second polypeptide sequence can include rhodopsin and portions or functional derivatives of rhodopsin. The second polypeptide can include cytochrome p450 and portions or functional derivatives of cytochrome p450. The second polypeptide can include a detectable protein label. Examples of contemplated detectable protein labels include, but are not limited to directly detectable protein labels, such as green fluorescent protein, and enzymic protein labels, wherein a substrate or product of a reaction catalyzed by the enzymic label is a detectable reporter agent. An illustrative example of an enzymic label is horseradish peroxidase. Functional portions of above indicated detectable protein labels are also contemplated.

In various preferred embodiments of the first aspect of the invention, the second polypeptide retains biological activity when incorporated in the chimeric capsid protein. The chimeric capsid protein, wherein the second polypeptide sequence retains biological activity, can bind to a nucleic acid. The chimeric capsid protein can bind to specified nucleic acid sequences. The chimeric capsid protein can bind to DNA. The chimeric capsid protein can bind to nucleic acids with specified structures, examples of which include, but are not limited to, double-stranded structures, single-stranded structures and regulatory element sequences and structures.

In various preferred embodiments of the first aspect of the invention, the second polypeptide binds to an antigen. In a further preferred aspect, the second polypeptide is an antibody. In another preferred embodiment, the second polypeptide is a protease.

In various preferred embodiments of the first aspect of the invention, the second polypeptide contains amino acid sequence derived from a necessary protein whose function is required to prevent, cure or ameliorate a diseased state. It is further contemplated that the necessary protein is a protein which is not present at adequate levels or for which its function is defective in a subject suffering from a diseased state. The necessary proteins contemplated include, but are not limited to, alpha glucosidase, glucocerebrosidase, glucose-6-phosphatase, atp7b protein and uridine diphosphate glycosyl transferase. It is also contemplated that the necessary protein may be a protein which is not required at the levels required to prevent, cure or ameliorate a diseased state in a subject not suffering from a diseased state or a predisposition towards a diseased state.

In various preferred embodiments of the first aspect of the invention, the second polypeptide is a nuclease. Nucleases contemplated include, but are not limited to, endonucleases, exonucleases, deoxyribonucleases and ribonucleases.

In various preferred embodiments of the first aspect of the invention, the second polypeptide is cytotoxic. It is contemplated that the second polypeptide is greater than 5, 10, 15, 25, 50, 75 or 100 amino acid residues in length. It is further contemplated that the second polypeptide contains the functional domains of protein toxins, including, but not limited to, the catalytic domain of diphtheria toxin.

In each of the various preferred embodiments of the first aspect of the invention, it is contemplated that the biological activity or function of the chimeric capsid protein may differ from that of either the first polypeptide sequence or the second polypeptide sequence or from either of the proteins from which the first polypeptide sequence or the second polypeptide sequence were derived. For example, a cytotoxic chimeric capsid protein containing a first polypeptide sequence and a second polypeptide sequence, neither of which, in and of themselves, are cytotoxic, is contemplated.

In a second aspect, the invention relates to a capsid which contains a chimeric capsid protein which contains a first polypeptide sequence and a second polypeptide sequence, wherein the first polypeptide sequence consists of native capsid protein amino acid sequence and the second polypeptide sequence consists of a heterologous non-capsid amino acid sequence. The second polypeptide sequence comprised in the chimeric capsid protein is displayed on the inner surface of the phage or viral capsid formed from the capsid protein.

In various preferred embodiments of the second aspect of the invention, the only capsid protein is the chimeric capsid protein of the first aspect of the invention. In additional preferred embodiments, the capsid comprises both the chimeric capsid protein of the first aspect of the invention and further capsid proteins. In a particular embodiment, the further capsid proteins include a protein from which the first polypeptide sequence of the chimeric capsid protein was derived.

In various preferred embodiments of the second aspect of the invention, the capsid is unenveloped. In another preferred embodiment, the capsid is isometric. In another preferred embodiment, the capsid forms without packaging nucleic acid. In a further preferred embodiment, nucleic acid encoding the capsid proteins can be physically occluded from the interior of the capsid or nucleic acid encoding the capsid protein can be not physically occluded from the interior of the capsid.

In a third aspect, the invention relates to a repetitive, ordered structure which contains capsids formed from the chimeric capsid protein which contains a first polypeptide sequence and a second polypeptide sequence, wherein the first polypeptide sequence consists of native capsid protein amino acid sequence and the second polypeptide sequence consists of a heterologous non-capsid amino acid sequence. The second polypeptide sequence comprised in the chimeric capsid protein is displayed on the inner surface of the phage or viral capsid formed from the capsid protein.

In various preferred embodiments of the third aspect of the invention, the capsids form a two-dimensional array or a three-dimensional array. In further preferred embodiments, the capsid can be immobilized on a solid support, a membrane, a lipid monolayer or a lipid bilayer.

In a fourth aspect, the invention relates to a nucleic acid which contains a transcriptional unit (TU) for a chimeric capsid protein. The TU directs the synthesis of the chimeric capsid protein, which contains a first polypeptide sequence and a second polypeptide sequence, wherein the first polypeptide sequence consists of native capsid protein amino acid sequence and the second polypeptide sequence consists of a heterologous non-capsid amino acid sequence.

In a preferred embodiment of the fourth aspect of the invention, the nucleic acid directs the synthesis of the chimeric capsid protein in vitro, in isolated cells, in cell culture, in tissues, in organs or in organisms. In other preferred embodiments, the nucleic acid is RNA or DNA. In a further preferred embodiment, the nucleic acid is a phagemid.

In a preferred embodiment of the fourth aspect of the invention, the nucleic acid contains a first region of nucleic acid sequence at the 5′ end of the nucleic acid sequence encoding heterologous amino acid sequence that specifies a first restriction endonuclease cleavage site and contains a second region of nucleic acid sequence at the 3′ end of the nucleic acid sequence encoding heterologous amino acid sequence that specifies a second restriction endonuclease cleavage site. In further preferred embodiments, the first and the second restriction endonuclease cleavage sites are for the same or are for different restriction endonucleases.

In a fifth aspect, the invention relates to the process of determining the structure of a polypeptide including the steps of: generating an isolated nucleic acid vector containing a transcriptional unit encoding a chimeric capsid protein of the first aspect of the invention, wherein the transcriptional unit directs the synthesis of the chimeric capsid protein; expressing the chimeric capsid protein encoded by the nucleic acid vector; forming capsids containing the expressed chimeric capsid protein; forming higher order arrays containing the capsids, namely repetitive ordered structures; obtaining x-ray diffraction patterns of the higher order arrays; and determining an atomic level or near-atomic level structure of the capsids, or a portion of the capsids, wherein the structure obtained includes the structure of the heterologous polypeptide.

In a preferred embodiment of the fifth aspect of the invention, the capsids containing the chimeric capsid protein also contain wild-type capsid protein. In another aspect, the higher order arrays, the repetitive ordered structures, of the capsids are two or three-dimensional arrays, including, but not limited to, crystals of the capsids. In another embodiment, determining an atomic level or near-atomic level structure of the capsids, or of a portion of the capsids, includes generating an electron density difference map between a crystal of wild-type capsid proteins and a crystal of chimeric capsid proteins. In another embodiment, determining a structure of the capsids, or a portion of the capsids, includes generating an electron density difference map between a crystal of a capsid of known structure and a crystal of chimeric capsid proteins of unknown structure. In further preferred embodiments, determining an atomic level or near-atomic level structure includes the use of a structure of the heterologous non-capsid amino acid sequence or the structure of a wild-type capsid protein as a search model to determine the structure of the chimeric capsid proteins.

In a sixth aspect, the invention relates to a method of characterizing the chimeric capsid proteins which consists of crystallizing capsids formed of the chimeric capsid proteins and analyzing the crystallized capsids.

In a preferred embodiment of the sixth aspect of the invention, the crystallization occurs in hanging drops using a vapor diffusion method. In another preferred embodiment, the crystallization occurs in volumes of solution whose composition is altered by dialysis, including, but not limited to the particular method of, microdialysis. In another preferred embodiment, the analyzing is by diffraction of electromagnetic radiation or particles, including, but not limited to the diffraction of x-ray radiation and neutrons.

In a seventh aspect, the invention relates to a method of identifying ligands of the chimeric capsid protein, which consists of contacting potential ligands of the chimeric capsid protein with the chimeric capsid protein under conditions whereby a ligand/protein complex can form and detecting ligand/protein complex formation. Detection of ligand/protein complex formation provides an indication that the potential ligand is bound by the chimeric capsid protein and, therefore, is a ligand of the chimeric capsid protein.

In an eighth aspect, the invention relates to a method of characterizing ligands of a chimeric capsid protein, which consists of contacting ligands of the chimeric capsid protein with the chimeric capsid protein, thereby forming a ligand/protein complex, forming capsids of the ligand/protein complex, and analyzing the crystallized capsids. The invention further relates to the related method wherein the chimeric capsid proteins are contacted with the ligands after formation of the crystallized capsids.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

For instance, one advantage of the chimeric capsid protein crystallization (Trojan Phage Crystallization System) described herein is that a single set of crystallization conditions, defined by the requirements for crystallization of the parent virus or phage capsid, results in the crystallization of one or more heterologous proteins thereby allowing structure determination. This is a significant advantage over current approaches and methods for protein crystallization, as crystallization of a set of heterologous protein sequences normally requires the determination, by empirical methods, of a separate set of crystallization conditions for each protein. Even if a set of suitable crystallization conditions may be found for each separate protein to be tested, it requires a large amount of time and effort which is often prohibitive. The use of the chimeric capsid protein described herein overcomes this shortcoming in the current art by providing a defined exterior, the external surface of capsids of chimeric capsid proteins, which allows the effective use of the same crystallization conditions for all chimeric protein molecules derived from a selected capsid protein sequence.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 is a schematic representation of a viral polyprotein encoded by a transcriptional unit according to the invention. In this particular example, a number of capsid proteins-are expressed as a single polyprotein which is processed to yield the individual proteins. VP4, VP2, VP3 and VP1 are native (wild-type) capsid proteins. “Target gene” is heterologous non-capsid amino acid sequence. The “protein shell precursor” is the polyprotein prior to processing. The “VP-target fusion protein” is a chimeric capsid protein in accordance with the invention.

FIG. 2 is a schematic representation of the structure of the assembled protomer, pentamer and capsid formed from the chimeric capsid protein of the invention. In this particular example, the protomer is formed from native VP2, VP3, VP4 and the chimeric capsid protein (VP1+Target Protein). Shown in the representation of the protomer, the heterologous non-capsid amino acid sequence is positioned on the surface of the assembled protomer and the assembled pentamer; formed from five protomers. In this particular example, twelve pentamers combine to form a single capsid. Also, as shown in the figure, the heterologous amino acid sequence of the chimeric capsid protein lies on the inner surface of the capsid (the position of the Target Protein is represented by a dotted circle to indicate its interior position).

FIG. 3 is a schematic diagram of HBV capsids formed by HBV core protein-S. aureus nuclease (HBV-SA) and HBV core-green fluorescence protein (HBV-GRF) chimeric capsid proteins. Addition of a heterologous SA nuclease domain to the carboxy terminus of the core protein results in formation of a capsid containing the chimeric capsid protein wherein the heterologous domain lies on the inner surface of the viral capsid formed from the HVB-SA nuclease capsid protein. Addition of a heterologous GRF domain to a region in the middle of the core protein results in formation of a capsid containing the chimeric capsid protein wherein the heterologous domain lies on the outer surface of the viral capsid formed from the HBV-GFP capsid protein. (FIG. 3 adapted from Beterams et al., FEBS Letters 481: 169-176 (2000)).

FIG. 4 is a schematic of pLCPCR1 construction. Restriction sites NotI and NspV were used to clone the PCR1 fragment containing regulatory elements T7/lac/p10 and RBS into pTriEx-1.1 to form pLCPCR1.

FIG. 5 is a schematic of pLCPCR2 construction. Restriction sites KpnI and NspV were used to clone the PCR1 fragment containing regulatory elements T7/lac/p10 and RBS into pTriEx-1.1 to form pLCPCR2.

FIG. 6 is a schematic of pLCPCR3 construction. Restriction sites AscI and PstI were used to clone the PCR1 fragment containing regulatory elements T7/lac/p10 and RBS into pTriEx-1.1 to form pLCPCR3.

FIG. 7 is a schematic of pLCVP4 construction. Restriction sites EcoRV and BglII were used to clone the rVP4PCR fragment containing the coding sequence for the structural capsid protein VP4 into pTriEx-1.1 to form pLCVP4.

FIG. 8 is a schematic of pLCVP1 construction. Restriction sites SpeI and NotI were used to clone the rVP1PCR fragment containing the coding sequence for the structural capsid protein VP1 into pLCPCR1 to form pLCVP1.

FIG. 9 is a schematic of pLCVP3 construction. Restriction sites NheI and NspV were used to clone the rVP3PCR fragment containing the coding sequence for the structural capsid protein VP3 into pLCPCR2 to form pLCVP3.

FIG. 10 is a schematic of pLCVP2 construction. Restriction sites NarI and PstI were used to clone the rVP2PCR fragment containing the coding sequence for the structural capsid protein VP2 into pLCPCR3 to form pLCVP2.

FIG. 11 is a schematic of pLCVP1t construction. Restriction sites BglII and NspV were used to clone the rTERVP1PCR fragment containing the termination signal sequence into pLCVP1 to form pLCVP1t.

FIG. 12 is a schematic of pLCVP2t construction. Restriction sites EcoRV and AscI were used to clone the rTERVP2PCR fragment containing the termination signal sequence into pLCVP2 to form pLCVP2t.

FIG. 13 is a schematic of pLCVP3t construction. Restriction sites EcoRV and KpnI were used to clone the rTERVP3PCR fragment containing the termination signal sequence into pLCVP3 to form pLCVP3t.

FIG. 14 is a schematic of pLCVP4-VP2t construction. The Bgl II-Pst I digestion fragment from pLCVP2t containing the VP2 expression cassette was cloned into pLCVP4 to produce a dual system, dual gene expression vector, pLCVP4-VP2t with target genes VP4 and VP2.

FIG. 15 is a schematic of pLCVP4-VP2t-VP3t construction. The PstI-NspV digestion fragment from pLCVP3t containing the VP3 expression cassette was cloned into pLCVP4-VP2t to produce a dual system, three gene expression vector, pLCVP4-VP2t-VP3t with target genes VP4, VP2 and VP3.

FIG. 16 is a schematic of pLCVP4-2t-3t-1t construction. The HindIII-NotI digestion fragment from pLCVP1t containing the VP1 expression cassette was cloned into pLCVP4-VP2t-VP3t to produce a dual system, four gene expression vector, pLCVP4-2t-3t-1t with target genes VP4, VP2, VP3 and VP1.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific molecular biological methods, specific viral or phage constructs or species, to specific heterologous proteins or to particular methods of structural determination, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a capsid protein” includes mixtures of capsid proteins, reference to “an expression vector” includes mixtures of two or more such vectors, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not. For example, the phrase “optionally mutagenized sequence” means that the sequence may or may not be mutagenized and that the description includes both wild-type and mutagenized sequence where there is mutation.

“Agent,” as used herein, means a molecule or species. Generally, agent will refer to a molecule or species with specific characteristics or properties which define the agent. Alternatively, an agent may be a molecule or species which potentially may possess specific characteristics or properties.

“Antibody,” as used herein, means a polyclonal or monoclonal antibody. Further, the term “antibody” means intact immunoglobulin molecules, chimeric immunoglobulin molecules, or Fab or F(ab′)₂ fragments. Such antibodies and antibody fragments can be produced by techniques well known in the art which include those described in Harlow and Lane (Antibodies: A Laboratory Manual Cold Spring Harbor Laboratory, Cold Spring Harbor, N.Y. (1989)) and Kohler et al. (Nature 256: 495-97 (1975)) and U.S. Pat. Nos. 5,545,806, 5,569,825 and 5,625,126, incorporated herein by reference. Correspondingly, antibodies, as defined herein, also include single chain antibodies (ScFv), comprising linked V_{H} and V_{L} domains and which retain the conformation and specific binding activity of the native idiotype of the antibody. Such single chain antibodies are well known in the art and can be produced by standard methods. (see, e.g., Alvarez et al., Hum. Gene Ther. 8: 229-242 (1997)). The antibodies of the present invention can be of any isotype IgG, IgA, IgD, IgE and IgM.

“Antigen,” as used herein, includes substances that upon administration to a vertebrate are capable of eliciting an immune response, thereby stimulating the production and release of antibodies that bind specifically to the antigen. Antigen, as defined herein, includes molecules and/or moieties that are bound specifically by an antibody to form an antigen/antibody complex. In accordance with the invention, antigens may be, but are not limited to being, peptides, polypeptides, proteins, nucleic acids, DNA, RNA, saccharides, combinations thereof, fractions thereof, or mimetics thereof.

Conditions whereby an antigen/antibody complex can form as well as assays for the detection of the formation of an antigen/antibody complex and quantitation of the detected protein are standard in the art. Such assays can include, but are not limited to, Western blotting, immunoprecipitation, immunofluorescence, immunocytochemistry, immunohistochemistry, fluorescence activated cell sorting (FACS), fluorescence in situ hybridization (FISH), immunomagnetic assays, ELISA, ELISPOT (Coligan, J. E., et al., eds. 1995. Current Protocols in Immunology. Wiley, N.Y.), agglutination assays, flocculation assays, cell panning, etc., as are well known to the person of skill in the art.

“Bind,” as used herein, means the physical association between a first and a second species. For example, as used herein, means the well-understood binding of a ligand by a receptor, an antigen by an antibody, a nucleic acid by a nucleic acid binding protein and so forth. “Specifically bind,” as used herein, describes an interaction between a first and a second species which is further characterized in that the nature of the binding is such that an antibody, a receptor or a nucleic acid binding protein binds their respective binding partner, but do not bind other species to a substantial degree. The nature of a binding reaction's specificity is contemplated to include the varying scope or character of species bound specifically by a binding partner, as is understood by those of skill in the art.

“Capsid,” as used herein, includes the shell-like structure of protein(s) which normally bounds and encloses the nucleic acid of bacteriophages, phages and viruses. Capsid, as used herein, also means structures derived from capsid proteins which do not bound nor enclose the nucleic acid of bacteriophages, phages or viruses. In particular, capsids formed from chimeric capsid proteins of the invention may form structures which occlude nucleic acids. Capsids formed from chimeric capsid proteins can have identical, similar or different external morphology.

A “chimeric protein” is a protein composed of a first amino acid sequence substantially corresponding to the sequence of a protein or to a large fragment of a protein (20 or more residues) expressed by the species in which the chimeric protein is expressed and a second amino acid sequence that does not substantially correspond to an amino acid sequence of a protein expressed by the first species but that does substantially correspond to the sequence of a protein expressed by a second and different species of organism. The second sequence is said to be foreign to the first sequence. The second sequence is also said to be a heterologous sequence in respect to the first sequence.

“Derived polypeptide” or “polypeptide derived from,” as used herein, means a peptide comprising or containing amino acid sequence, structure, function or immunoreactivity derived from a selected polypeptide, protein or antigen. Examples include, but are not limited to, polypeptides of sequence corresponding to; a selected antigen or a fragment of a selected antigen; a selected enzymic label or a fragment of a selected enzymic label; a selected nucleic acid binding protein or a fragment of a selected nucleic acid binding protein; a selected antibody or a fragment of a selected antibody; a selected protease or a fragment of a selected protease; a selected necessary protein or a fragment of a selected necessary protein; a selected nuclease or a fragment of a selected nuclease; or a selected toxin or a fragment of selected toxins.

“Detectable protein labels” means both a protein, or a portion thereof, which is itself detectable, or which generates a detectable signal itself, and a protein, or portion thereof, which allows modification of the protein to allow detection. Therefore, examples of a detectable protein label include, but are not limited to, fluorescent, radioactive, immunoreactive and enzymatically active proteins and functional portions thereof. Correspondingly, detectable protein labels can be detected by detection of an fluorescent or immunofluorescence moiety (e.g., green fluorescent protein, or by detection using fluorescein- or rhodamine-labeled antibodies against an antigen contained in the chimeric capsid protein), a radioactive moiety (e.g., ³²P, ¹²⁵I, ³⁵S), an enzyme moiety (e.g., horseradish peroxidase, alkaline phosphatase), a colloidal gold moiety, an avidin moiety and a biotin moiety. (see, e.g., Harlow and Lane, Antibodies: A Laboratory Manual, Cold Spring Harbor Laboratory, Cold Spring Harbor, N.Y. (1989); Yang et al., Nature 382:319-324 (1996)).

“Envelope,” as used herein, means an encompassing structure or membrane. Specifically, it refers to the well-known meaning as the term is used in virology, namely, the coat surrounding the capsid and usually furnished at least partially by the host cell. Correspondingly, an unenveloped virus or phage includes any virus or phage lacking an envelope, including phage or viral constructs derived from enveloped species which have been engineered, modified or treated to either prevent formation of an envelope or to remove an envelope.

“Isometric,” as used herein to describe phage and viruses, means that the phage or viruses are built up on the structural principles known to those of skill in the art which give isometric viruses roughly spherical shapes.

“Membrane,” as used herein, means both the well understood material of commerce and widespread use in the field of biotechnology and the well understood biological structures consisting largely of proteins and lipids. Which meaning of the term that is applicable for a given situation is to be understood by the context in which it is used and is within the discernment of one of skill in the art. Membrane, as the well understood material of commerce, also encompasses other flexible, non-rigid sheets of polymeric or elastomeric materials. Examples include, but are not limited to, nylon, nitrocellulose, or equivalent materials known to those of skill in the art. As described herein, a membrane can be used as a solid support. Membrane, as the well understood biological structure, means both any biologically derived membrane, such as that derived from cell membranes, and artificially produced facsimiles thereof as are known to those of skill in the art. Examples of closely related sheet-like, relatively fluid structures include lipid bilayers and lipid monolayers.

“Mimetic,” as used herein, includes a chemical compound, or an organic molecule, or any other mimetic, the structure of which is based on or derived from a binding region of an antibody or antigen. For example, one can model predicted chemical structures to mimic the structure of a binding region, such as a binding loop of a peptide. Such modeling can be performed using standard methods. In particular, the crystal structure of peptides and a protein can be determined by X-ray crystallography according to methods well known in the art. Peptides can also be conjugated to longer sequences to facilitate crystallization, when necessary. Then the conformation information derived from the crystal structure can be used to search small molecule databases, which are available in the art, to identify peptide mimetics which would be expected to have the same binding function as the protein (Zhao et al., Nat. Struct. Biol. 2: 1131-1137 (1995)). The mimetics identified by this method can be further characterized as having the same binding function as the originally identified molecule of interest according to the binding assays described herein.

Alternatively, mimetics can also be selected from combinatorial chemical libraries in much the same way that peptides are. (Ostresh et al., Proc. Natl. Acad. Sci. USA 91: 11138-11142 (1994); Dorner et al., Bioorg. Med. Chem. 4: 709-715 (1996); Eichler et al., Med. Res. Rev. 15: 481-96 (1995); Blondelle et al., Biochem. J. 313: 141-147 (1996); Perez-Paya et al., J. Biol. Chem. 271: 4120-6 (1996)).

“Monocistronic,” as described herein, can mean that the referred to sequence, RNA or DNA, encodes for a single protein or polypeptide. Similarly, “bicistronic” indicates that the referenced sequence encodes for two proteins or polypeptides. Other such terms derived from the root “cistronic” will, of course, be clear and definite in meaning to those of skill in the art of molecular biology and other related arts such as described herein or which circumscribe the subject matter of the present invention.

“Necessary protein,” as described herein, means a protein whose presence and function is necessary to prevent, cure or ameliorate a diseased state. “Diseased state, in this context, refers to the normally understood meaning of the term, namely, the state of any deviation from or interruption of the normal structure or function of any body part, organ or system that is manifested by a characteristic set of symptoms and signs and whose etiology, pathology and prognosis may be known or unknown. Further, as used herein, “diseased state” refers to a state wherein physical, mental and social well-being are not maximized.

A coding sequence is “operably linked” to a promoter, enhancer or other regulatory region (e.g., when both the coding sequence and the promoter together constitute a native or recombinant gene) when the promoter is capable of directing transcription of the coding sequence, when the enhancer is capable of increasing transcription of the coding sequence or when the regulatory region is capable of affecting the transcription of the coding sequence.

As used herein, the term “origin of replication” (ORI), can mean the general starting point or origin of replication of the genetic material of eukaryotic or prokaryotic cells and viruses. Methods for the isolation of ORI sequences are well-known to those of skill in the art as is the testing of putative ORI sequences for activity in cells of desired types. Such methods are described by DePamphilis in Annu. Rev. Biochem 62: 29-63 (1993). Typical methods used include, but are not limited to, for example, “nascent strand extrusion” (Kaufmann et al. Mol. Cell. Biol. 5: 721-727 (1985)) and “anticruciform immunoaffinity purification” (Bell et al., Biochem. Biophys. Acta. 1089: 299-308 (1991)).

“Phage” or “bacteriophage,” as used herein, relates to the well-known category of viruses of bacteria. “Virus,” as used herein, means the well-understood term of the art, as well as other species which may be derived from phage and viruses as are understood and known by those of skill in the art.

“Solid support,” as used herein, means the well-understood solid material to which various components of the invention are physically attached, thereby immobilizing the components of the present invention. The term “solid support,” as used herein, means a non-liquid substance. A solid support can be, but is not limited to, a membrane, sheet, gel, glass, plastic or metal. Immobilized components of the invention may be associated with a solid support by covalent bonds and/or via non-covalent attractive forces such as hydrogen bond interactions, hydrophobic attractive forces and ionic forces, for example.

Reference will now be made in detail to the present preferred embodiments of the invention, examples of which are illustrated in the accompanying drawings. Wherever possible, the same reference numbers are used throughout the drawings to refer to the same or like parts.

The invention encompasses nucleic acids which contain transcriptional units that encode chimeric capsid proteins. The nucleic acids function to direct the expression of the chimeric capsid proteins of the invention. The expression of the chimeric capsid protein(s) can be in vitro, namely, in cell-free protein expression systems (as described in U.S. Pat. No. 6,238,884 and references cited therein), in isolated cells, in cell culture, in tissues, in organs or in organisms. The nucleic acid may be a plasmid or vector encoding additional genes or particular sequences for the convenience of the skilled worker in the fields of molecular biology and virology (See “Molecular Cloning: A Laboratory Manual,” 2^{nd} Ed., Sambrook, Fritsch and Maniatis, Cold Spring Harbor Laboratory, Cold Spring Harbor, N.Y., 1989; and “Current Protocols in Molecular Biology,” Ausubel et al., John Wiley and Sons, New York 1987 (updated quarterly)), which are incorporated herein by reference). Other aspects relating to the expression of viral or phage proteins or the construction of suitable vectors can also be found in U.S. Pat. Nos. 6,057,098, 6,177,075 and references therein, which are hereby also incorporated by reference.

The nucleic acid molecules of the instant invention designate nucleic acids, or functional derivatives of nucleic acids, whose nucleotide sequence encode specific gene products including, among other encoded proteins or functional nucleic acids, chimeric capsid proteins, the nucleic acids may encode further proteins. The further proteins may be capsid proteins. In an important embodiment, the nucleic acids are DNA. Alternatively, the nucleic acids are RNA. The nucleic acids may also be any one of several derivatives of DNA or RNA whose backbone phosphodiester have been chemically modified to increase the stability of the nucleic acid. Modifications so envisioned include, but are not limited to, phosphorothioate derivatives or phosphonate derivatives; these and other suitable modifications are well-known to those of skill in the art of nucleic acid chemistry.

An important nucleic acid containing a transcriptional unit encoding chimeric capsid proteins of the instant invention is a DNA. In order to function effectively, it is advantageous to include, within the nucleic acid, a control sequence that has the effect of enhancing or promoting the translation of the sequences encoding the chimeric capsid proteins. Use of such promoters is well known to those of skill in the fields of molecular biology and genetic engineering (“Molecular Cloning: A Laboratory Manual,” 2^{nd} Ed., Sambrook, Fritsch and Maniatis, Cold Spring Harbor Laboratory, Cold Spring Harbor, N.Y., 1989; and “Current Protocols in Molecular Biology,” Ausubel et al., John Wiley and Sons, New York 1987 (updated quarterly) ). It will be recognized the optimal nucleic acid sequences to be used as promoters, as well as other regions of sequence, will depend upon the system of protein expression used, namely, the particular embodiments of the invention used in prokaryotic host cells or in eukaryotic host cells will require different sequences, each adapted for use in the specific host cells to be used in the practice of the invention. For instance, if expression is to be carried out in eukaryotic cells, use of a cytomegalovirus early promoter is contemplated.

In an important embodiment, the nucleic acid of the invention is any phagemid suitable for the practice of the invention as would be recognized by one of skill in the art (Hoogenboom et al., Nucl. Acids Res. 19: 4133-4137 (1991)). In particular, the phagemid can be constructed so that the only viral components encoded or expressed are protein capsid or shell components, thereby rendering the construct noninfectious. By way of illustrative example, the phagemid like construction will be a circular DNA molecule which contains the genes for the picomavirus capsid proteins, a bacterial and/or phage replication origin and at least one selection marker, such as, but not limited to ampicillin, kanamycin, etc. The bacterial and/or phage replication origins will allow the construct to be propagated in bacterial cells, such as, but not limited to, E. coli. Propagation in bacterial cells can be used for the synthesis, construction, manipulation and amplification of the nucleic acid. Optionally, the expression of proteins can be carried out in bacterial or other prokaryotic cells. Optionally, the nucleic acid of the invention can also include eukaryotic replication origins and/or promoters allowing the expression of chimeric or wild-type, ie., native, capsid proteins in eukaryotic cells. Expression of proteins in either the prokaryotic or eukaryotic cells of the invention can be used for capsid assembly. It is contemplated that the genes for capsid protein be under the control of an inducible promoter as is known to those of skill in the art.

Nucleic acids of the invention may be constructed using the standard techniques of the field of molecular biology using the known nucleic acid and protein sequences available to those of skill in the art.

It is contemplated that the nucleic acids of the invention be constructed so that a first region of nucleic acid sequence at the 5′ end of the nucleic acid sequence which encodes the heterologous sequence comprises a first restriction endonuclease cleavage site and that a second region of nucleic acid sequence at the 3′ end of the nucleic: acid sequence encoding a heterologous amino acid sequence specifies a second restriction endonuclease cleavage site. Construction of a nucleic acid of the invention in this preferred manner allows the excision of one particular heterologous sequence and introduction a second particular heterologous sequence at the same site in accordance with the standard molecular biology techniques of those of skill in the art. It is further contemplated that the first and second restriction endonuclease sites be such that they are cleavage sites for either the same or for two different restriction endonucleases. It is further contemplated that the restriction endonuclease sites are part of a multiple cloning site or a region of sequence like a MCS.

As contemplated herein, the nucleic acid of the invention can be constructed so as to contain one, two, three or more multiple cloning sites (MCS). Each MCS will include multiple restriction endonuclease cleavage sites, thereby allowing the heterologous amino acid sequence (aka, the target protein, the non-capsid protein sequence, the second polypeptide) expressed to be easily altered or changed by altering, replacing or changing the nucleic acid sequence cassette which encodes that sequence of the chimeric capsid protein of the invention. It is further contemplated that the first, second and any further MCSs can include restriction endonuclease sites that are unique thereby allowing excision of sequence from those sites having the unique restriction endonuclease sites and not from any other site not having the unique or nearly unique restriction endonuclease site.

In a particular respect, viral capsid proteins of capsids of known structure, or the corresponding viral nucleocapsid, are selected for the practice of the invention. Alternatively, the native capsid protein amino acid sequence selected may be from a capsid of unknown structure. Examples of capsids of known structure, wherein the structure has been determined to high resolution, include those listed in Table One. Use of these capsids for the practice of the invention is preferred.

The structures of some of the preferred viral capsid proteins for use in the construction and practice of the invention include those that have been solved to a resolution between 1.8 and 4 Å. Sizes of preferred viral capsids for use in the construction and practice of the invention include those of greater than 20, 30, 40, 50, 60, 70, 80, 90, 100, 120, 140, 160, 180, 200, 225 and 250 nm in diameter are preferred.

The use of capsids and capsid proteins for which the capsids are isometric are contemplated. In particular, those capsids which are icosahedral and which display cubic symmetry are preferred. It is further preferred that the capsids be derived from unenveloped phage or viruses.

In a particular respect, heterologous amino acid sequence may be selected from any protein or amino acid sequence which is heterologous in respect to the native capsid protein. It is contemplated that proteins with a specific activity, or portions of proteins which confer a specific activity, will be used as a source of heterologous amino acid sequence. In each case, functional derivatives of the selected protein are also encompassed by the present invention. Chimeric capsid proteins, and capsids formed from same, specifically contemplated include all or a portion of rhodopsin and cytochrome p450. In one particular aspect, rhodopsin in formed capsids is contemplated for use as an information storage cell in accordance with the teachings of Lewis et al., Science 275: 1462-1464 (1997)). In one particular aspect, it is contemplated that a chimeric capsid protein comprising cytochrome p450 be used to a therapeutic agent in the detoxification of tissues and/or samples or other substances or mixtures. In a particularly useful aspect, it is contemplated that capsids of chimeric capsid proteins having detoxification activity be directed to the liver of subjects suffering from toxification or be used ex vivo to provide detoxification of tissues which may be removed from subjects and then returned including, but not limited to, blood and lymph.

Proteins comprising a detectable protein label are also contemplated. As used herein, a detectable protein label is any portion of a protein that can be specifically detected when expressed. Detectable protein labels are useful for detecting or quantitating expression of a protein and are useful for localizing the position of an expressed protein. Many detectable protein labels are known to those of skill in the art. These include, but are not limited to, horseradish peroxidase, β-galactosidase, luciferase, and alkaline phosphatase that produce specific detectable products. Fluorescent reporter proteins can also be used, such as green fluorescent protein (GFP), cyan fluorescent protein (CFP), red fluorescent protein (RFP) and yellow fluorescent protein (YFP). For example, by utilizing GFP, fluorescence is observed upon exposure to ultraviolet light without the addition of a substrate. The use of a reporter proteins that, like GFP, are directly detectable without requiring the addition of exogenous factors are preferred for detection of a specified chimeric capsid protein.

Chimeric capsid proteins which bind nucleic acids are also encompassed by the invention. Specific examples contemplated include, but are not limited to, RNA binding proteins, such as the Rev protein, an HIV associated regulatory RNA binding protein that facilitates the export of unspliced HIV pre mRNA from the nucleus (see, e.g., Malim et al., Nature 338:254 (1989)); DNA binding proteins, such as single stranded dna binding protein (SSB) or any of the DNA binding proteins comprising one or more zinc finger motifs, leucine zipper motifs, helix-turn-helix motifs, or a combination thereof. In related respects, the nucleic acid binding proteins will include those which bind to specific structures and it will include those that bind to specific sequences. It is further contemplated that some chimeric capsid proteins of the invention will bind to regulatory elements, such as, but not limited to, attenuators, operators, promoters and repressors.

Chimeric capsid proteins which bind to antigens, especially those chimeric capsid proteins containing antibodies, are contemplated. Antibodies, or functional fragments or derivatives thereof, can be produced in accordance of the invention, by; presenting antigen or a fragment thereof to an immune system, generating polyclonal antibodies, selecting the single B cell which produces an antibody of interest, using the single, selected B cell to produce a hybridoma, determining the functional amino acid sequence of the antibody from the hybridoma and generating a chimeric capsid protein wherein the heterologous non-capsid amino acid sequence comprises the functional amino acid sequence of the antibody.

As is known to those of skill in the art, an antibody to an antigen of choice can be produced according to Kohler and Milstein, Nature, 256:495-497 (1975), Eur. J.

Immunol. 6:511-519 (1976), both of which are hereby incorporated by reference, by immunizing a host with the antigen of choice. Once a host is immunized with the antigen, B-lymphocytes that recognize the antigen are stimulated to grow and produce antibody to the antigen. A collection of the sera containing the antibodies produced by these B-lymphocytes contains the disclosed antibodies that can be used in the disclosed methods.

Each activated B-cell, produces clones which in turn produce the monoclonal antibody. B-cells cannot be cultured indefinitely, however, and so a hybridoma must be produced. Hybridomas are produced using the methods developed by Kohler and Milstein, Nature, 256:495-497 (1975). Hybridomas can be produced by fusing the B-cells obtained by the host organism's spleen to engineered myeloma cells. These cells often have a selectable marker which prevents them from growing in a medium, if they have not been fused to a B-cell. Likewise, B-cells are not immortal and so those that are unfused will die. Thus, the only cells left after fusion are those cells which have come from a successful B-cell and myeloma cell fusion. The fusion cells are analyzed to determine if the desired antibody is being produced by a given fused cell, by for example, testing the fused cells with the antigen in an ELISA assay. The antibodies produced and isolated by this method are specific for a single antigen or epitope on an antigen.

In another embodiment of the invention, it is contemplated the second polypeptide contains amino acid sequence from a protein, a necessary protein, whose function is required to prevent, cure or ameliorate a diseased state. It is further contemplated that the necessary protein be a protein which is not present at adequate levels or is defective in function in a subject suffering from a diseased state. By way of non-limiting examples, only for the sake of illustrating the principle, a necessary protein for a subject suffering from: phenylketonuria would be phenylalanine-4-monooxygenase; hemophilia A would be Factor VIII; and so forth. It is further contemplated that the necessary proteins be proteins for which the necessary protein is not required at the levels required to prevent, cure or ameliorate a diseased state in a subject not suffering from a diseased state or a predisposition towards a diseased state. Particular disease states and necessary proteins contemplated include, but are not limited to: Refsum disease (incorrect lipid metabolism) and peroxisomol phytanoyl-CoA alpha hydroxylase (PHYH) (Genebank accession number AAB81834); Gyrate atrophy of the choroids (elevated levels of ornithine in plasma) and ornithine amino transferase (Genebank accession number CAA68809); Zellweger syndrome (improper protein sorting) and peroxisomal targetting signal receptor 1 (Genebank accession number AAC50103); phenylketonuria (PKU) and phenylalanine hydroxylase (Genebank accession number AAA60082); and Amyotrophic Lateral Sclerosis (Lou Gehrig Disease) and superoxide dismutase 1 (SOD1) (Genebank accession number CAA26182). Further necessary proteins specifically contemplated for treatment of any of the lysosomal diseases, such as Gaucher's disease, Tay-Sachs disease, Cystinous or Pompe's disease, include alpha glucosidase, glucocerebrosidase, glucose-6-phosphate, atp7b protein and uridine diphosphate glycosyl transferase.

In another embodiment of the invention, it is contemplated that the second polypeptide is a nuclease or functional portion or derivative thereof. It is further contemplated that the resulting chimeric capsid protein retain nuclease activity. Specific types of nuclease encompassed in the invention include endonucleases, exonucleases, deoxyribonucleases and ribonucleases. Further, the specificity of the nucleases from which the selected heterologous non-capsid amino acid sequence is derived may be for single-stranded nucleic acid (for example, but not limited to, S1 nuclease and ribonuclease T1) or it may be for double-stranded nucleic acid (for example, but not limited to, EcoRI or ribonuclease V1). In a particularly preferred example, the nuclease is S. Aureus nuclease (Beterams et al., FEBS Letters 481: 169-176 (2000)).

In another embodiment of the invention, it is contemplated that the second polypeptide be a cytotoxic polypeptide. It is further contemplated that the chimeric capsid protein be toxic and/or comprise a toxin. Toxins are poisonous substances produced by plants, animals, or microorganisms that, in sufficient dose, are often lethal. Diphtheria toxin is a substance produced by Corynebacterium diphtheria which can be used therapeutically. This toxin consists of an alpha and beta subunit which under proper conditions can be separated. It is further contemplated that ricin be used to generate a cytotoxic chimeric capsid protein. In this specific example, the alpha-peptide chain of ricin, which is responsible for toxicity, is selected as the second polypeptide of the chimeric capsid protein.

Many peptide toxins have a generalized eukaryotic receptor binding domain; in these instances the toxin must be modified to prevent intoxication of cells not bearing the targeted receptor. In one embodiment of the present invention, it is contemplated that the chimeric capsid protein provide selectivity for the spatial or temporal delivery of toxins to cells or tissues. Any modifications made to the toxin when constructing the chimeric capsid protein of the invention are preferably made in a manner which preserves the cytotoxic functions of the molecule. Potentially useful toxins include, but are not limited to: cholera toxin, ricin, Shiga-like toxin (SLT-I, SLT-II, SLT-IIV), LT toxin, C3 toxin, Shiga toxin, pertussis toxin, tetanus toxin, Pseudomonas exotoxin, alorin, saporin, modeccin, and gelanin. Diphtheria toxin can be used to produce chimeric capsid proteins useful as described herein. Diphtheria toxin, whose sequence is known, and hybrid molecules thereof, are described in detail in U.S. Pat. No. 4,675,382 to Murphy. The natural diphtheria toxin molecule secreted by Corynebacterium diphtheriae consists of several functional domains which can be characterized, starting at the amino terminal end of the molecule, as enzymatically-active Fragment A (amino acids Gly1-Arg193) and Fragment B (amino acids Ser194-Ser535), which includes a translocation domain and a generalized cell binding domain (amino acid residues 475 through 535). While the normal process by which diphtheria toxin intoxicates sensitive eukaryotic cells will differ from what normally occurs for an unmodified diphtheria toxin, the following description of the process by which unmodified diphtheria toxin acts will provide to one of skill in the art a basis for understanding the function of the chimeric capsid protein of the invention: (i) the binding domain of diphtheria toxin binds to specific receptors on the surface of a sensitive cell; (ii) while bound to its receptor, the toxin molecule is internalized into an endocytic vesicle; (iii) either prior to internalization, or within the endocytic vesicle, the toxin molecule undergoes a proteolytic cleavage between fragments A and B; (iv) as the pH of the endocytic vesicle decreases to below 6, the toxin crosses the endosomal membrane, facilitating the delivery of Fragment A into the cytosol; (v) the catalytic activity of Fragment A (i.e., the nicotinamide adenine dinucleotide—dependent adenosine diphosphate (ADP) ribosylation of the eukaryotic protein synthesis factor termed “Elongation Factor 2”) causes the death of the intoxicated cell. A single molecule of Fragment A introduced into the cytosol is sufficient to inhibit the cell's protein synthesis machinery and kill the cell. The mechanism of cell killing by Pseudomonas exotoxin A, and possibly by certain other naturally-occurring toxins, is very similar.

While not wishing to be bound by theory, it is believed that selection of an appropriate capsid from which to derive capsids of chimeric capsid proteins will allow targeting of chimeric capsid protein containing the a functional portion of diphtheria toxin into the cytosol of specific targeted cells, thereby causing the death of the specific targeted cell. It will be further understood by one of skill in the art, that similar aspects of the invention which do not cause the death, but which have an effect consistent with the delivered toxin or agent, are within the scope of the invention.

In certain embodiments, it is contemplated that the enzymatically active domains of these toxins may be used as the heterologous non-capsid amino acid sequence of the present invention. It is specifically contemplated that the enzymatically active A subunit of E. coli Shiga-like toxin be utilized (the toxin is described in Calderwood et al., Proc. Natl. Acad. Sci. USA 84:4364 (1987) and its use in a hybrid is described in U.S. Pat. No. 5,906,820). The enzymatically active portion of Shiga-like toxin, like diphtheria toxin, acts on the protein synthesis machinery of the cell to prevent protein synthesis, thus killing the cell.

It is contemplated that the localization of the toxin in the interior of a formed capsid will lessen undesirable aspects of the toxins used and that the ability to use capsids which can target delivery to specific cells or cell types will increase the efficacy and specificity of the resulting cytotoxic capsid protein. The use of the current invention to lessen undesirable side-effects of toxicity, to reduce the quantity of toxins required, and to increase the tissue and or/cell specificity of a treatment using a toxin are specifically contemplated. It is further contemplated that the second polypeptide of the chimeric capsid protein be greater than 5, 10, 15, 25, 50, 75 and 100 amino acids in length.

In particular embodiments, the design of the encoded chimeric capsid protein is facilitated by the use and analysis of the known structures of viral or phage capsids. These structures may be obtained from the Brookhaven National Laboratory Protein Database or any other suitable repository or may be determined in the practice of the invention.

As will be recognized by one of skill in the art, insertion, addition or substitution of heterologous amino acid sequence in the capsid protein is preferred at positions in the native capsid protein which lie on the inner surface of the native capsid. It is contemplated that the practice of the invention can include a visual inspection and analysis of viral capsid protein structures and selection of an appropriate capsid protein and position in the amino acid sequence of the protein for the insertion of a heterologous amino acid sequence. In some instances, it will be recognized that deletion of native capsid sequence will be required and, that in other instances, deletion of native capsid sequence will not be required. These particular aspects of the invention's practice will depend upon the particular capsid, capsid protein and heterologous amino acid sequence chosen; these particular aspects will be recognized to be within the range of abilities of one of skill in the art and are recognized to not amount to undue experimentation. In particular, it will be recognized that the design of chimeric capsid proteins take into account the structural aspects and domains of both the native capsid protein and the heterologous non-capsid protein.

Criteria to be considered in designing the chimeric capsid protein to be expressed, in particular, in the choice of where to join the native capsid amino acid sequence and the heterologous amino acid sequence, include, but are not limited to: choice of where in primary, secondary, tertiary and quaternary structure that the two proteins be joined to form a splice junction. It is preferred, in many instances, that the splice junction be made at either the amino or carboxy terminus of at least one of the proteins from which sequence is derived to form the chimeric capsid protein, as this simplifies subcloning procedures (e.g., it only necessitates maintaining the correct reading frame through a single splice junction). It is preferred, in many instances, that the splice junction be located on the inner surface of the capsid formed containing the chimeric capsid protein, in other words, it is preferred that the target protein be incorporated on the interior of the viral shell. It is preferred, in many instances, that the splice junction be located at a region which is centrally located in the protomer units that are formed from the chimeric capsid protein. It will be appreciated by those of skill in the art that the placement of a splice junction and/or a target protein sequence near the edges of the protomer unit may be more likely to interfere with capsid assembly than a more centrally located placed splice junction or target protein as the edges directly interact with other protomers during capsid assembly.

It will be understood by those skilled in the art that the proteins of the invention that are recombinantly or synthetically combined to produce the chimeric capsid proteins of the invention specifically include amino acid sequences containing conservative amino acid substitutions of the foregoing sequences. In such sequences, one or a few amino acids of one or more of the foregoing amino acid sequences are substituted with different amino acids having highly similar properties. The replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

It will also be recognized by one of skill in the art that “conservatively modified variations” of a particular nucleic acid sequence, nucleic acids which encode identical or essentially identical amino acid sequences, or where the nucleic acid does not encode an amino acid sequence, are within the scope of the invention. Because of the degeneracy of the genetic code, a large number of functionally identical nucleic acids encode any given peptide. Such nucleic acid variations are silent variations, which are one species of conservatively modified variations. One of skill will recognize that each codon in a nucleic acid (except AUG, which is ordinarily the only codon for methionine) can be modified to yield a functionally identical molecule by standard techniques. Accordingly, each silent variation of a nucleic acid which encodes a peptide is implicit in any described amino acid sequence. Furthermore, one of skill will recognize that, as mentioned above, individual substitutions, deletions or additions which alter, add or delete a single amino acid or a small percentage of amino acids (typically less than 5%, more typically less than 1%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (1), Leucine (L), Methionine (M), Valine (V); and

6) Phenylalanine (F), Tyrosine (Y), Tryptophan (W).

Further, it will be recognized by one of skill in the art that two polynucleotides or polypeptides are said to be “identical” if the sequence of nucleotides or amino acid residues in the two sequences is the same when aligned for maximum correspondence. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. Chimeric antigens can be produced by standard molecular biology techniques wherein a single nucleic acid is synthesized which encodes the chimeric antigen. Nucleic acids that encode chimeric antigens can be produced by recombinant procedures by ligation of synthetic or recombinant nucleic acids to produce a single nucleic acid that encodes a chimeric antigen and the recombinant nucleic acid is used to direct the synthesis of the desired chimeric antigen in a cell or cell extract. Alternatively, the nucleic acid that directs the synthesis of the chimeric antigen may be synthesized chemically and used to direct the synthesis of the desired chimeric antigen in a cell or cell extract. These methods are well known in the art and are described further in Maniatis et al., “Molecular Cloning: A Laboratory Manual” (1989), 2^{nd} Ed., Cold Spring Harbor, N.Y.; Berger et al., Methods in Enzymology, Volume 152 and “Guide to Molecular Cloning Techniques” (1987), Academic Press, Inc., San Diego, which are incorporated herein by reference.

If expression of the encoded proteins is to be carried out in prokaryotic cells, use of prokaryotic specific promoters and control elements is contemplated. If expression of the encoded proteins is to be carried out in eukaryotic cells, use of eukaryotic specific promoters and control elements is contemplated. Illustrative examples of useful systems for the expression of capsids and capsid proteins can be found in U.S. Pat. Nos. 242,426; 6,217,870; 6,218,180; 6,204,044; 6,177,075; 6,132,732; and 5,916,563. These examples are not intended to limit the ways in which the nucleic acid of the invention is obtained, but to provide illustrative examples for one of skill in the art.

It is contemplated that the transcriptional unit containing nucleic acid molecules of the instant invention may be introduced into appropriate cells in many ways well known to skilled workers in the fields of molecular biology and viral immunology. By way of example, these include, but are not limited to, incorporation into a plasmid or similar nucleic acid vector which is taken up by the cells, such as a phagemid, or encapsulation within vesicular lipid structures such as liposomes, especially liposomes comprising cationic lipids, or adsorption to particles that are incorporated into the cell by endocytosis.

In general, a cell of this invention is a prokaryotic or eukaryotic cell comprising a nucleic acid of the invention or into which the nucleic acid has been introduced. A suitable cell is one which has the capability for the biosynthesis of the encoded products as a consequence of the introduction of the nucleic acid. In particular embodiments of the invention, a suitable cell is one which responds to a control sequence and to a terminator sequence, if any, which may be included within the nucleic acid. In order to respond in this fashion, such a cell contains within it components which interact with a control sequence and with a terminator and act to carry out the respective promoting and terminating functions. When the cell is cultured in vitro, it may be a prokaryote, a single-cell eukaryote or a multicellular eukaryote cell. In particular embodiments of the present invention, the cell is bacterial, yeast, insect or mammalian cell.

In an illustrative embodiment, recombinant baculoviruses are produced which encode the phage or viral capsid protein, or a chimeric capsid protein. To form capsids and/or proteins of the invention, host insect cells (for example, Spodoptera frugiperda cells) are either infected with recombinant baculoviruses encoding all capsid proteins necessary for formation of capsids, are coinfected with recombinant baculoviruses encoding the chimeric capsid protein and any other required capsid protein or after expression of proteins, the proteins are isolated and combined under conditions wherein capsid formation occurs.

In a preferred embodiment of the invention, in vitro translation systems or cells and nucleic acids of the invention are used such that the capsids self assemble. The assembled capsids are isolated therefrom.

In further aspects, as will be recognized by those of skill in the art, the chimeric capsid proteins provide pentamers and/or other structures formed from the capsid proteins which are not fully formed or intact capsids. In particular, it is contemplated that the invention provide the intermediate structures formed with the chimeric capsid proteins of the invention which are further combined to form capsids.

In further aspects, as will be recognized by those of skill in the art, the chimeric capsid proteins provide capsids containing the chimeric capsid proteins of the invention. In certain aspects, the capsids provided by the invention may be such that the only capsid protein present is the chimeric capsid protein. It is also contemplated that the capsids of the invention may also contain other capsid proteins. These other capsid proteins can be either the capsid protein from which the chimeric capsid protein is derived or they can be other capsid proteins.

It is contemplated that in some aspects of the invention, the capsid be unenveloped. It is contemplated that in some aspects the capsid be isometric. It is contemplated that in some aspects, the capsid have a generally icosohedral shape. It is contemplated that in some aspects, the capsids have a filamentous shape.

It is further contemplated that the capsids of the invention can be formed without packaging of nucleic acid, particularly, without packaging the nucleic acid molecules which encode the proteins from which the capsids of the invention are formed. It is further contemplated that, of the capsids formed in accordance with the invention, some will, and some will not, physically occlude the nucleic acid encoding the capsid protein or proteins of the capsid from the interior of the capsid.

It is further contemplated that the capsids of the invention can be arranged to form or can form repetitive ordered structures. By way of non-limiting example, if a capsid of the invention was constructed using capsid protein sequence from tobacco mosaic virus coat protein, crystals comparable to the crystals of tobacco mosaic virus are provided by the invention (see U.S. Pat. No. 5,618,699).

It is further contemplated that capsids of the invention can form a two-dimensional array. This array can include the aspect that the capsids be immobilized on a solid support. It is further contemplated that the capsids may be immobilized on a membrane, a lipid monolayer or a lipid bilayer. An example of such an array, not formed of the chimeric capsid proteins of the invention, but which still illustrates these principles, has been described (McDermott et al., J. Mol. Biol. 302: 121-133 (2000)).

It is further contemplated that the capsids can form a three-dimensional array. This array can include the aspect that the capsids be immobilized on a solid support. It is further contemplated that the capsids be immobolized on a membrane, a lipid monolayer or a lipid bilayer. Examples of such arrays, not formed of the chimeric capsid proteins of the invention, but which still illustrate these principles, have been described (Yusibov et al., J. Gen. Virol. 77: 567-573 (1996); U.S. Pat. Nos. 6,090,609 and 5,714,374, and references contained therein).

In another aspect, the invention provides a process for the determining the structure of a polypeptide. In one aspect, the process includes the steps of: generating a nucleic acid of the invention which directs the synthesis of a chimeric capsid protein of the invention; forming capsids containing the chimeric capsid proteins; forming a repetitive ordered array containing the capsids; obtaining x-ray diffraction patterns using the repetitive ordered array to diffract x-rays; and determining an atomic, or near-atomic, level structure of the polypeptide. As will be recognized by the foregoing description of this process of the invention, each step of the process besides obtaining x-ray diffraction patterns of the repetitive, ordered arrays and determination of the structure has been described in detail above.

An illustrative description of a process to obtain the x-ray diffraction patterns and to determine the structure of each portion of the structure, including the polypeptide comprised in the structure, of which can be used in the practice of the invention is described along with the method of electron density averaging (Kleywegt et al., Structure 5: 1557-1569 (1997); Vellieux et al. in Methods in Enzymology 277: 18-53, Carter and Sweet eds., Academic Press, Orlando (1997)).

It is also contemplated that the capsids from which the structure is derived may contain only chimeric capsid proteins an both chimeric capsid proteins and native capsid proteins. It is further contemplated that not all of the capsids in the ordered, repetitive array be of identical composition. It is further contemplated that the ordered, repetitive arrays may be crystals.

It is further contemplated that the process of determining a structure will further comprise the use of a structure of a heterologous non-capsid amino acid sequence, the structure of a wild-type capsid protein or the known structure of a chimeric capsid protein to determine the structure of a chimeric capsid protein.

In another aspect, the invention provides a method of characterizing the chimeric capsid proteins, consisting of crystallizing capsids formed of the chimeric capsid proteins of the invention and analyzing the crystallized capsids. It will be appreciated by those of skill in the art that the crystallization of proteins or other molecules of interest can be of great use in the determination of structures. In a specific manner of use, it will be recognized that crystallizing capsids of chimeric capsid proteins can be a significant aid in the determination of the 3-dimensional structures of proteins or protein domains when using x-ray diffraction analysis.

The crystalline form is one in which many molecules of the protein are aligned with each other. This presentation of the protein molecules delivers a strong signal in an X-ray diffraction unit. It will be recognized that incorporated protein sequences or the specific binding or complex formation of other molecules to the incorporated protein sequences of the chimeric capsid proteins of the invention are aligned with respect to one another by the ordered structures formed by the capsids of the invention. It should be further recognized that the use of the x-ray crystallographic, or other related techniques, will allow clearer and more detailed structures of the heterologous amino acid sequences incorporated into the chimeric capsid proteins or to molecules specifically associated with the chimeric capsid proteins, such as, but not limited to, nucleic acids, drugs, metabolites and the like.

Crystallization of the capsids of the invention can be carried out according to the standard practices of those of skill in the art. As the external dimensions and characteristics of the capsids of the invention are unaltered, or only slightly altered, in respect to the analogous non-chimeric capsid, or different chimeric capsids, and, as the external dimensions and characteristics of capsids dominate other factors influencing crystallization, the crystallization of chimeric capsids can be carried out according to the methods used for crystallization of the phage or viral capsids from which they are derived, or according to methods but slightly altered from the methods known in the art. While more specific and detailed protocols are known to, or readily determined by, those of skill in the art, general techniques contemplated include, but are not limited to, crystallization in hanging drops using vapor diffusion and crystallization in volumes of solution whose composition is altered by microdialysis. A list of viruses and phage suitable for the practice of the invention, along with a summary of suitable crystallization conditions with the outcome of crystallization and references describing the method of crystallization, are included in Table 2.

The crystals containing capsids containing chimeric capsid proteins can be analyzed by using the crystals to diffract electromagnetic radiation or particles, such as, but not limited to x-rays and neutrons. Examples of methods and protocols for the practicing this aspect of the invention may be found throughout the references incorporated herein, particularly those relating to the crystallization and structure determination listed in Table 2.

In another important aspect, the current invention provides methods of identifying ligands of the chimeric capsid protein. In these methods, the chimeric capsid proteins can be contacted with agents or potential ligands under conditions which allow the formation of a complex between the agent or potential ligand and the chimeric capsid protein of the invention and then detecting the presence of the formed complex, thereby determining that the potential ligand or agent is bound by the chimeric capsid protein. Examples of potential ligands or agents include, but are not limited to, small molecules, peptides, proteins, nucleic acids, and derivatives or mimetics thereof.

It will be recognized that the methods for screening potential ligands or agents to identify compounds which interact with and bind to the chimeric capsid proteins of the invention can vary. For example, the chimeric capsid protein may be in an isolated form in solution, or in immobilized form, as an isolated, single protein, as a pentamer, as a capsomer, as an capsid. For example, the potential ligands or agents may similarly be in isolated form in solution or in immobilized form. Regardless of the form of the chimeric capsid protein, a plurality of compounds are contacted with the chimeric capsid protein under conditions sufficient to form a complex. Alternatively, the method can be altered to screen for agents or ligands which inhibit the formation of complexes between species which normally form complexes with a chimeric capsid protein of the invention and a chimeric capsid protein of the invention.

Once an agent or ligand has been identified which interacts with a chimeric capsid protein of the invention, the use of the chimeric capsid protein crystallization system may be used to characterize the nature of the interaction or interactions responsible for stabilizing the interaction. As will be recognized by those of skill in the art, contacting ligands or agents with chimeric capsid proteins and forming complexes of the ligands or agents with the chimeric capsid proteins, followed by crystallization of capsids containing the chimeric capsid proteins and the solution of the structure of the chimeric capsid protein with bound ligand or agent provides a structure of the ligand or agent bound to the chimeric capsid protein.

Further aspects of the invention that relate to vectors, proteins derived from vectors and the use of vectors that comprise multiple monocistronic expression units that effect expression in both at least one prokaryotic and at least one eukaryotic cell type are contemplated. These are further described herein.

In preferred embodiments of the present invention, in particular, vectors of the invention, the origin of replication is functional in the recombinant host cell of choice. Preferably, an origin of replication is function in more than one type of host cell such that the vector can be replicated in more than one type of host cell. For example, an origin of replication is functional in both a prokaryote cell and a eukaryote cell. Alternatively, the vectors of the invention comprise multiple ORI wherein at least one is function in at least one prokaryotic cell type and at least one is functional in at least one eukaryotic cell type.

Transcriptional expression of desired genes encoded by the vectors of the invention can be accomplished by incorporation of transcription control signals such as, but not limited to promoters and transcriptional enhancers and, if polypeptide synthesis from the encoded sequence is desired, efficient translational start and stop sequences. One particular translational start sequence that is effective for supporting efficient translational expression of a gene in both eukaryotes and prokaryotes can include a minimum sequence of GCC or ACC. This minimum sequence, when placed immediately upstream of the translational start codon of the gene to be expressed, promotes efficient translation. A preferred translation start codon is AUG. Inclusion of GCC or ACC just ahead of the AUG supports efficient protein synthesis in E. coli and other prokaryotes; in yeast, mammalian cells, in Xenopus laevis cells and other eukaryotes; and in protozoans such as Leishmania. As is known in the art, the sequence (GCC)GCCRCCAUGG (SEQ ID NO:1), where R is adenine or guanine, is an optimal sequence for protein expression (Kozak, J. Mol. Biol. 196: 947-950 (1987)). Other particularly preferred sequences for promoting efficient protein expression are described in U.S. Pat. No. 5,874,242, incorporated herein by reference for the teachings relating to translational start site design. In particular, the design of the UTE sequences described therein are contemplated for use in selected vectors of the present invention. The LacZ.ACC-NcoI-KpnI-AgeI oligonucleotide, namely, the sequence of TAAGG ATCCT TAACA CAGGA GGCAG ACCAT GGTAC CGGT (SEQ ID NO:2), described in U.S. Pat. No. 5,874,242 can be used. Alternatively, other sequences can be used, including, but not limited to others selected from the Ner gene of bacteriophage Mu.

More particularly, promoters are understood to be nucleic acid sequences that regulate the transcription of a gene. Normally, promoters are 5′ to the gene sequence regulated. Enhancers are also understood to be useful in the function of the invention. Enhancers can function as activators of gene expression. Unlike promoters, enhancers can be further from the gene and can be either 5′ or 3′ of the gene of which transcription is increased. Sequences that repress gene activity can also be useful in the function of the invention. Inclusion of repressor sequences can be used to down-regulate the level of expression of particular sequences in accordance with the needs as known to those of skill in the art.

Promoters, enhancers and repressors can be constitutive, cell-specific, cell-cycle specific, tissue-specific, metabolically-regulated, and/or inducible. Certain preferred promoters include composite promoters derived from T7 promoter, lac operon sequence and p10 promoter (as described in the Examples). This promoter, built from three promoter elements; two prokaryotic promoter elements, the T7 promoter and the lac operon, and one eukaryotic promoter element, the p10 promoter can express genes in both prokaryotic and eukaryotic cells. This promoter can share more than, less than, or approximately, 75, 80, 85, 90, 95 or 100% DNA sequence homology with the sequence described in the Examples. Another preferred promoter is any promoter that substantially corresponds to promoters for Chlorella virus DNA methyltransferase genes. These promoters can share more than, less than, or approximately 75, 80, 85, 90, 95 or 100% DNA sequence homology with the Chorella virus DNA methyltransferase promoter as described in U.S. Pat. No. 6,252,140 which is incorporated herein by reference for its teachings regarding the promoter and variants thereof. In preferred embodiments, the promoters can function in both prokaryotic and eukaryotic cells. These promoters can support high levels of constitutive expression of heterologous genes.

Expression of two or more genes is a useful aspect of the present invention. The vector makes possible to coordinated expression of two or more genes in a single cell from a single vector. In certain aspects the coordinated expression is control so that the ratio of a first expressed sequence or product thereof (i.e., an expressed polypeptide) and a second expressed sequence or product thereof is greater than 0.01, 0.05, 0.1, 0.2, 0.3, 0.5, 0.8, and 1. In other aspects, the ratio of the first expressed sequence or product thereof and a second expressed sequence or product thereof is less than 1, 0.8, 0.5, 0.3, 0.2, 0.1, 0.05, and 0.01. In alternative embodiments, the vector can express greater than 2, 3, 4, 5, or 6 separate, noncontiguous sequences. In alternative embodiments, the vector can express less than 7, 6, 5, 4, or 3 separate, noncontiguous sequences. The sequences expressed can be mono-, bi-, or tri-cistronic. Particularly preferred embodiments are vectors comprising monocistronic transcriptional units that express RNAs encoding a single protein construct. The characterization of a mRNA as monocistronic does not preclude that the monocistronic RNA comprises sequence encoding portions of greater than one polypeptide, but rather that the expressed peptide sequence is a single polypeptide.

Selection and optimization of promoter, enhancer, repressor elements and the like can be used to adjust or modulate the level of expression. Further, translational control elements can also be used to adjust or modulate the level of expression. In certain embodiments, it is contemplated that the level of a first and the level of a further gene product can be altered to effect proper function and/or structure. For example, maintenance of proper stoichiometric ratios between subunits that combine to form multimeric functional units is contemplated. It is further contemplated that the stoichiometric production of subunits can be utilized for the preparation of of those subunits by cultivating host cells which are transformed with the expression vectors according to the invention in suitable media and separating the combination of subunits, i.e., multimeric units comprising subunits, produced from the cells and the medium. In particular, the present invention provides for the expression of proteins encoded by the first, second and, optionally, any further genes comprised in the vectors of the present invention.

Media for use in this aspect, and related aspects, of the present invention can include any known media for cultivating the particular cell used as a host cell. If, for example, the host cell is a mammalian cell, the media can be selected from the group including synthetic, protein-free or protein poor media such as, but not limited to, DMEM (Dulbeco's modified Eagle medium), enriched with 4.5 g/L glucose and 5 to 10% FCS thereby facilitating both protein production and purification of expressed protein from the growth media.

Marker gene expression can be used to screen for the presence or absence of the vectors of the invention. Further, use of selective pressure, such as, but not limited to, the inclusion of an antibiotic in growth media, can be used to ensure retention of a vector of the present invention. Specific examples of selectable marker genes are neomycin phosphotransferase gene, apramycin resistance gene, hygromycin β-phosphotransferase gene, dihydrofolate reductase gene, guanine phosphoribosyl transferase gene and thymidine kinase gene. Alternatively, a reporter gene can be used to indicate the presence of a vector of the invention. For example, a reporter gene can be a chloramphenicol acetyltransferase gene, β-galactosidase gene, β-glucuronidase gene or human growth hormone gene.

In particular aspects of the present invention, the vector is successfully retained following removal of selective pressure. For example, the vector can be successfully retained following at least 5, 10, 15, 20, 25, 30, 40, 50, 60, 80 or 100 generations. In other particular aspects of the invention, the vector is not successfully retained following removal of selective pressure. For example, the vector is not successfully retained following no more than 5, 10, 15, 20, 25, 30, 40, 50, 60, 80, 100 or 150 generations. A vector is considered to be successfully retained if it can still be detected by Southern blot analysis and/or only a small number of cells die in tissue culture after re-addition of selective pressure.

Vectors can include still further additional DNA sequences that provide appreciable benefits. These include, but are not limited to, DNA sequences that provide for easy selection, amplification, and transformation in prokaryotic and eukaryotic cells. The additional DNA sequences include, as mentioned earlier, origins of replication to provide for autonomous replication of the vector, selectable marker genes preferably encoding antibiotic resistance, unique multiple cloning sites providing for multiple sites to insert heterologous sequences, and sequences that enhance the transformation of prokaryotic and/or eukaryotic cells. In certain aspects, the vector can include or exclude sequence from bacterial, yeast, animal, mammalian and plant vectors. For example, any portion up to and including the entire vector can exclude any sequence from any of the afore-mentioned vectors. Tᵢ vector sequence can be included or excluded from the present invention. Any Agrobacterium vector, or portion thereof, can be excluded. For example, nopaline T-DNA left and right borders can be excluded. Vectors can be derived from and/or include sequence from virus vectors. Such virus vectors include, but are not limited to, M13 phage system vectors, the vaccinia virus system vectors, and the baculovirus system vectors. A virus promoter, mutant and/or fragment thereof can be combined with further DNA sequence to form DNA constructs that allow or facilitate expression of the desired gene sequence in both prokaryotic and eukaryotic cells.

A vector according to the present invention can be an expression vector. The expression vectors can express genes of differing types in different host cell types. Genes expressed can be under the control of host-specific gene regulatory sequences. Alternatively, the genes expressed can be under the control of non-host specific gene regulatory sequences. That is, the gene regulatory sequence can be a heterologous regulatory sequence not normally found in the host cell. If the gene regulatory sequence is heterologous, any necessary cofactors can be added to the host cell to allow regulatory sequence function. If cofactors are added, they can be added by inducing their production from other heterologous nucleic acid sequence and/or they can be added by addition to media comprising the host cells. Other heterologous nucleic acid sequence that supports the production of necessary cofactors can be included in the same vector as the genes expressed or can be on different nucleic acid sequence (e.g., on a separate vector). Normally, the expression vector for expression of the gene sequence will comprise promoter, operator and terminator sequences for the transcription and the sequence of ribosomal binding sites for the translation. Expression vector such as these are suitable for in vitro, ex vivo and in situ expression of various genes in both eukaryotes and in prokaryotes.

Specific examples of genes that can be expressed include, but are not limited to plant cell genes, bacterial genes, eukaryotic genes, mammalian genes, and synthetic genes. Genes to be expressed can also be viral genes for structural proteins, including viral genes for chimeric constructs of structural proteins that further comprise heterologous sequence.

An expression vector of the present invention can be introduced into prokaryotic and/or eukaryotic host cells to provide those cells with the capacity to express the desired genes encoded by the first and at least second DNA sequence under control of a first and second promoter. Cells containing the expression vector can express the genes encoded by the first and second DNA sequence. Gene expression in the cells can be detected by a variety of methods including oligonucleotide probe hybridization to mRNA, assay for the activity of the gene product (if present), or by detection of the gene products by their physical characteristics. The transformation can be either transient or stable. The expression can be either transient or stable. If transformation is stable, the expression vector can be found in daughter cells following cell reproduction. If transformation is transient, then the expression vector is lost from cells following cell reproduction.

Gene product expression can be constitutive. Gene product expression can be inducible. Gene expression in use of the present invention can be either constitutive or inducible. The type of expression used for each gene, i.e., the first, second and any further genes, can be either constitutive or inducible in nature.

In certain embodiments of the invention, an expression vector can be introduced into prokaryotic host cells. The preferred host cells for expression will vary with the characteristics of the vector of the invention in accordance with the teachings of the art. For example, the preferred host cells can be E. coli. An expression vector can be introduced into bacterial cells by standard methods including, but not limited to, use of calcium co-precipitation and electroporation. Transformed cells comprising an expression vector can be subjected to selective pressure to identify those that have been transformed. Alternatively, they can be identified by use of a detectable marker.

In certain embodiments of the invention, an expression vector can be introduced in eukaryotic cells, including, but not limited to, yeast and cultured plant or animal cells. An expression vector can be introduced into eukaryotic cells by use of calcium phosphate co-precipitation, protoplasting, electroporation, biolistic transformation or by Agrobacterium-mediated transformation.

As will be appreciated by those of skill in the art, the presence of a gene or a particular sequence can be determined by a variety of methods. For example, hybridization to oligonucleotide probes. Also, as will be appreciated, the expression of an encoded polypeptide can be detected by antibodies or, if the encoded peptide has a determinable activity, by assays for the determinable activity. Determination of the effectiveness or relative efficiency of expression at an RNA level can be determined by monitoring the level of RNA and comparing this to the level of DNA present. Determination of the effectiveness or relative efficiency of translation can be determined by monitoring the level of protein and comparing this to the level of RNA present.

In certain aspects, the vector of the invention is distinguished in that it contains two or more promoter sequences, operator sequences, and terminator sequences such that each set of promoter, operator and terminator sequences support the synthesis of RNA encoding messages. In certain aspects, the messages encode polypeptide sequence. In other aspects, the messages can encode functional nucleic acids. Examples of functional nucleic acids include ribozymes and antisense RNA molecules. Other functional nucleic acids include rRNAs and tRNAs. Each of these supported syntheses from the vector can constitute expression.

The vector of the invention, as will be recognized, can be derived by use of many different vectors that express a single encoded message. Such a derivation for one vector is described explicitly in the examples so as to provide a working example from which those of skill in the art can comprehend certain aspects of the invention. However, the present vectors of the invention are not limited to those derived from any one such vector. Other vectors can, of course, also be used, for example, pIRES vector (Clontech), pIRES2-EGFP vector (Clontech), pPUR vector (Clontech), pTet-On & pTet-Off vectors (Clontech), pTK-Hyg selection vector (Clontech) and others.

Alternatively, the derivation can be to modify vectors already expressing multiple monocistronic messages, such as for example, the pTriEx™ multisystem expression vectors (Novagen), by altering them so as to be effective expression vectors in both at least one prokaryotic cell type and at least one eukaryotic cell type. Such modification can include duplication of appropriate sequences required for expression in both prokaryotes or in eukaryotes and inclusion of these sequences into the vector. If replication of the vector is desired, i.e., not only transient expression of the expressed sequences is desired, appropriate ORIs can also be included.

Expression of the sequence encoded by the expression vector of the invention can be in vitro expression, ex vivo expression, in vivo expression, in situ expression and expression in cultured cells, tissues, organs, or organisms. In particular examples, the vector can be used for the expression of the encoded sequences for use in inducing the expression in organisms. If such expression is desired, the organism can be a transgenic organism comprising the vector in cells allowing heritable transmission from parent cells to daughter cells in the growing and/or reproducing organism. Alternatively, the vector can be delivered to cells in the organism thereby allowing transient expression. Either way, if the vector is transmitted to an organism, rather than a single cell or small number of cells that is allowed to develop into an organism, delivery of the vector can be accomplished using the methods known to those of skill in the art. In particular embodiments, those methods include use of parenteral administration.

Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLE ONE

Echovirus I (EV1) Phage Construct

Genetically engineered viral self-assembling chimeric capsid proteins for the crystallization and structure determination of macromolecules are prepared from an isolated nucleic acid comprising a transcriptional unit that encodes a chimeric capsid protein. In this particular example, the encoded chimeric capsid protein is the fusion formed by the addition of hen egg white lysozyme to capsid proteins of echovirus 1.

It is further contemplated that this system be automated, thereby making significant contributions to many proteomic and structure based drug design projects. In particular, providing the ability to grow crystals of any suitable target protein and to improve crystallization conditions for molecules that have intellectual, therapeutic and commercial value.

Echovirus 1 (EV1) self-assembling capsid proteins (VP1-4) are produced from an isolated nucleic acid encoding the capsid proteins and hen egg white lysozyme in accordance with the detailed description, U.S. Pat. No. 4,946,676 and the knowledge of the skilled practitioner of the art.

In certain respects, the first examples demonstrates the synthesis of the initial genetic constructs that encode the capsid proteins of the invention. Design of a the Chimeric Capsid Protein Crystallization System requires selection of the viral system to be genetically modified. Requirements to be used in selecting a system can include all or part of the following:

1. Known crystallization conditions. Using a viral system for which the capsids of the virus or phage are known to crystallize and for which there exists known crystallization and data collection parameters a priori reduces the work involved in optimizing the conditions to yield useful crystals for x-ray diffraction. Furthermore, prior data allows one of skill in the art to estimate of the limit of resolution obtainable.

2. Size. Small phage or viruses, if suitable, require the least effort in determining its structure and the structure of the interior positioned heterologous amino acid sequence. However, the selected phage or virus should be chosen so as to provide an internal volume adequate to provide accomodation of structure formed by the heterologous amino acid sequence.

3. Shape. The use of a spherically shaped virus, icosohedral or isometric virus, can aid in the structure determination of the chimeric capsid protein, particularly of the structure formed by the heterologous amino acid sequence using electron density averaging techniques already available.

4. Nonenveloped virus. Nonenveloped viruses are generally less complex and generally are more amenable to crystallization and structure determination. Correspondingly, nonenveloped viruses or phage are preferred in the practice of the invention.

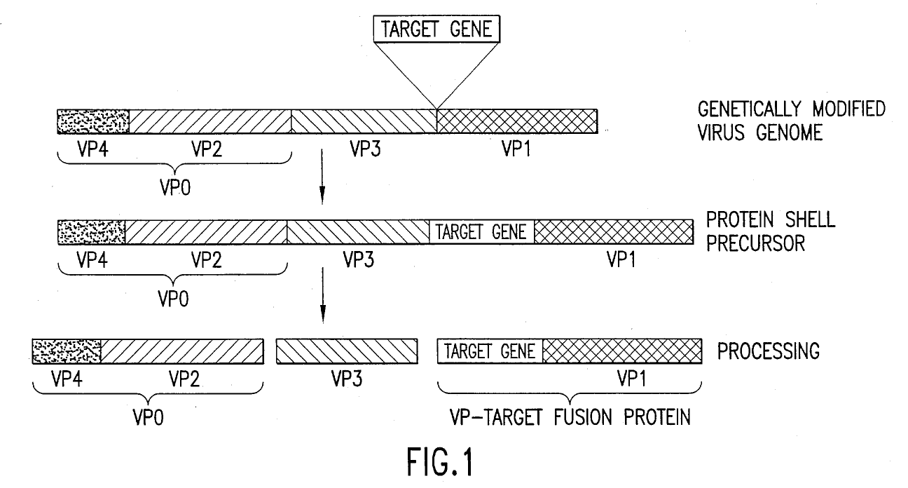

In accordance with the above indicated selection criteria, echovirus 1 (EV1) was selected for use in practicing the invention. Visual inspection of the EV1 and related viral capsid protein structures suggest that modification of protomer subunit VP1 may be a useful approach. However, as the capsid protomer is composed of four subunits (VP1-4), modification of each of the four subunits to incorporate heterologous sequence is contemplated. FIG. 1 illustrates the modification of VP1, ie., the construction of a chimeric capsid protein consisting of VP1 protein sequence and heterologous amino acid sequence. and is contemplated. The heterologous amino acid sequence, the test protein, chosen is hen egg white lysozyme. Lysozyme is a protein that has a well-known structure, crystallization conditions and is amendable to the theoretical volume and other size limitations in of this system as outlined in the criteria for selecting a system outlined above.

Construction of EV1 VP-lysozyme fusion proteins. The hen egg white lysozyme gene, encoding a 15 kD protein, is genetically fused in frame to VP1, 2 or 3. The target protein gene (lysozyme) is subcloned in frame to either the 5′ or 3′ termini of VP1, 2 or 3 using a linker sequence. Visual inspection of the structure of VP proteins from enteroviruses EV1, polio and coxsackie 3B indicates that fusion of the target protein to the amino terminus of native VP1 to form a chimeric capsid protein will not significantly interfere in the assembly of protomers or capsids, in other words, this fusion does not prevent subunit assembly. The amino terminus of the VP1 protein is located near the interior center of the protomer unit. Nucleic acid sequencing is used to ensure that the proper reading frame has been maintained throughout the chimeric capsid protein gene. The vector is designed specifically for propagation in prokaryotic cells for amplification, for DNA sequencing and for expression in eukaryotic cells for viral capsid production. Because these particles assemble without the incorporation of the viral genome they are not infectious in the commonly accepted meaning of the term, although they can cross the cell membrane and be internalized. Construction of full-length, in frame VP-lysozyme gene fusions as determined by DNA sequencing is followed by expressing the chimeric capsid proteins and the other capsid proteins required for assembly, if other capsid proteins are required.

Specifics of the design of the cloning and subcloning procedures are in accordance with the teaching of the art and the sequence of the EV1 genome (Genbank accession number AF029859), including the addition of appropriate genetic linker(s) to maintain the correct open reading frames for the encoded proteins. As the nucleic acid of the invention is also propagated as either a plasmid or a phagemid, other design criterion are incorporated such that promote amplification and selection in bacteria.

The DNA manipulations are the conventional routine laboratory protocols of the art. Amplification of small regions of DNA is performed using the polymerase chain reaction (PCR). All PCR products are sequenced to insure proper nucleotide incorporation.

The expression of complete chimeric capsid proteins, and other capsid proteins, required for the formation of capsid of chimeric capsid proteins is demonstrated using routine biochemical techniques. For instance, the expressed proteins are tested by SDS-PAGE and immunoblot analysis to demonstrate both that the expressed proteins are of the correct size and that the expressed proteins have the correct structure and/or function. For the chimeric capsid protein which is a VP1-lysozyme fusion, immunoreactivity with VP1-specific and lysozyme-specific antibodies demonstrates correct expression and adequate folding for at least some aspects of the invention.

The chimeric capsid proteins, expressed in relatively large amounts, are used in assembling capsids. The viral capsid proteins are self-assembling units that may be exploited for protein crystallography. The structure of the echovirus 1 (EV1), a member of the well characterized picornavirus family (Harrison et al., 1996)(Rossmann et al., 1985), has previously been determined by molecular replacement to 3.5 Å (Filman et al., 1998). The picornavirus family is characterized by small spherically shaped membrane un-coated viruses that have a single stranded RNA genome of approximately 7500 nucleotides. This family can be subdivided into enteroviruses, rhinoviruses, cardioviruses, aphthoviruses and hepatitis A virus genera. Echovirus as well as polio and coxsackie viruses belong to the enterovirus genera. Echovirus has a protein sequence similarity of 50% with polioviruses and 75% with coxsackievirus B3 (Filman et al., 1998). Expression, purification, crystallization and cryo-cooling conditions have been determined for the EV1viral crystals (Filman et al., 1998). The viral capsid of EV1 forms a shell with an outside diameter of 260 Å. This shell encapsulates the viral single strand RNA genome and functions in infection. The capsid is formed from 60 subunits called protomers. Each protomer is composed of four protein molecules (VP1, VP2, VP3 and VP4). The protein shell is 34 Å thick leaving an inside diameter of 192 Å.

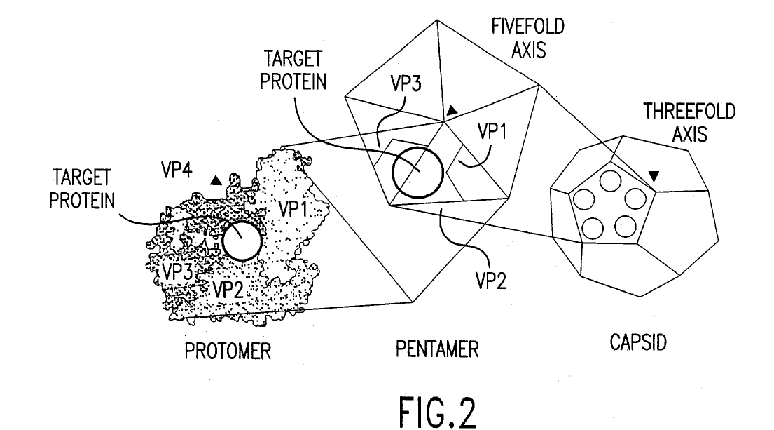

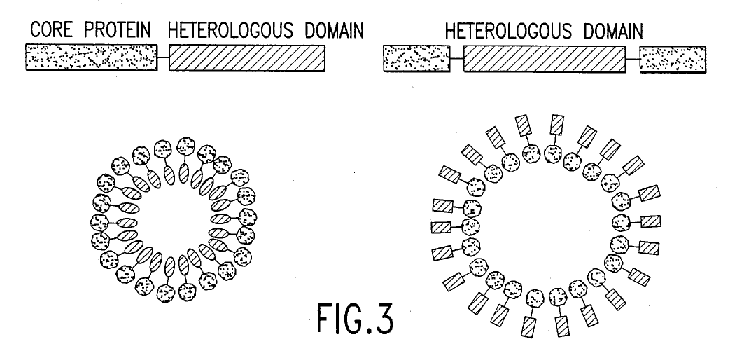

The chimeric capsid protein crystallization system is designed such that the chimeric capsid protein, in which the heterologous amino acid sequence, the target protein, is contained, is covalently linked to the interior surface of each one of the 60 capsid protomers (FIGS. 2, 3). During capsid self-assembly the VP-target protein fusion protomers are incorporated into the structure and display viral symmetry. The exterior of the capsid particle effectively mimics the native virus surface and hence crystallize under similar conditions as reported. That is, any protein displayed on the interior surface of the empty viral capsid submits to native virus structure crystallization conditions. Given the inside diameter and the ability to form the capsid void of genetic material results in a volume of 61734 Å³ available for each of the target proteins to occupy. As one Dalton (D) of protein occupies 1.228 Å³ (Matthews, 1968), this system could accommodate a protein of up to 50 kD in mass. These modified viral crystals diffract x-rays and the resultant patterns are interpreted and solved using molecular replacement and electron density modification techniques. The viral and crystal symmetries are used for density averaging techniques to improve the quality and interpretation of calculated electron density maps.

As the structure determination of molecules of up to 50 kD is a systematic procedure, it is amendable to high through-put proteomic projects.

In one manner of practicing the invention, the formation of empty picornavirus capsid particles for x-ray crystal analysis is achieved by the addition of guanidine-HCl. The efficient self-assembly of the enteroviruses is encoded in the tertiary structure of the viral capsid proteins VP1, VP2, VP3 and VP4. Protein molecules VP1, VP2 and VP3 are similar in size (ca. 30 kD) and share a common tertiary structural fold composed of an eight-stranded β-barrel fold. The VP4 molecule is smaller, at 7.5 kD. A single copy of each protein folds together to form the major building block of the capsid, called a protomer. The picornavirus viral shell displays icosahedral symmetry T=1, (P=3) that is built up from the assembly of 60 protomer units. Capsid assembly is driven by concentration gradients.

The picornavirus genome is translated from a single open reading frame that results in a large polyprotein with a size near 200 kD. The polyprotein is processed in a series of proteolytic steps that yield individual proteins. An early cleavage results in a 100 kD polyprotein (P1) that encodes for the capsid molecules. P1 is then cleaved twice to make VP1, VP3 and an immature capsid protein precursor, VP0. Late in the infection VP0 is cleaved to make VP2 and VP4. In a picornavirus infection a variety of capsid protein intermediates have been discovered. These include the P1 protomer, a cleaved protomer containing one copy of VP0, VP1 and VP3, a pentamer containing copies of VP0, VP1 and VP3, an empty capsid consisting of 60 copies of VP0, VP1 and VP3, and the mature virus which has the 60 copies of VP1, VP2, VP3, VP4 and a single RNA molecule. There is controversy over the role of the empty capsids in the virus assembly reaction. Pulse chase experiments are consistent with a pathway that produces pentamers that go on to form the empty shells. It appears that the proteolytic processing of VP0 into VP2 and VP4 is important for RNA internalization (Basavappa et al., 1994). The equilibrium for enhancing production of empty capsids can be shifted by adding millimolar quantities of guanidine-HCl that inhibits encapsulation of RNA. This shift in the formation of empty capsids allows for milligram quantities of virus to be produced and purified from infected eukaryotic HeLa cell monolayers.

Purification, crystallization, data collection and even structure determination by molecular replacement methods is practiced in accordane to those methods developed for EV1. Viral particles are purified by centrifuging clarified cell extracts through a 30% sucrose cushion and then through a CsCl density gradient. Particle concentration is performed by centrifugation through a 30% sucrose cushion made 1 M NaCl in buffer (10 mM PIPES, 5 mM MgCl₂, 1 mM CaCl₂, pH 7.0). Crystals are be grown by microdialysis against C buffer (10 mM PIPES, 25 mM CaCl₂, 25 mM MgCl₂, 2.5% PEG 400, pH 6.0) at 277 K (Filman et al., 1998). Viral crystals were cryo-protected by stabilization in 25% ethylene glycol in buffer C at 277 K, then transferred to 30% ethylene glycol and % glycerol in buffer C for 1 minute at 277 K prior to flash freezing. A complete data set can be collected from a single crystal on a rotating anode generator. Filman et al., collected data to 3.5 Å due to limitations in the recording system used but observed that diffraction occurred to at least 3.0 Å. This level of resolution can be enhanced with further optimization of crystallization conditions and the use of intense X-radiation from a synchrotron sources.

Plant viruses are used in one variation of the method. The use of plant viruses provide specific benefits due to the well-understood processes of protein maturation, capsid assembly and the ability to produce gram quantities of material (Johnson and Chiu, 2000; Oliveira et al., 2000; Canady et al., 2000).

The chimeric capsid protein crystallization system, as an ensured protein crystallization system, reduces and/or eliminates early bottlenecks in proteomic studies (Lamzin and Perrakis, 2000). This is a significant improvement to the art as current estimates of the rates of success without the use of the current invention are around 10%. These estimates also generally identify critical bottlenecks which hinder success. The major successes required to overcome the critical bottlenecks are the expression and purification of protein molecules and the crystallization of protein suitable for x-ray analysis. Current automated processes, the availability of intense x-radiation from synchrotron sources and improvements in calculating phases, either from molecular replacement or multiple anomalous dispersion (MAD) strategies, all appear able to handle large numbers of crystallized proteins for structure determination. It is the production of suitable crystallized proteins which have prevented the appropriate advance in x-ray structural proteomics. The system of the present invention produces the suitable crystallized proteins, in that the system can guarantee sufficient quantities of pure protein with known crystallization parameters. Correspondingly, application of the system would alleviate the immediate bottlenecks foreseen during current proteomic projects.

References

Basavappa, R., Syed, R., Flore, O., Icenogle, J. P., Filman, D. J. and Hogle, J. M. Role and mechanism of the maturation cleavage of VP0 in poliovirus assembly: structure of the empty capsid assembly intermediate at 2.9 Å resolution. Protein Science (1994) 3: 1651-1669.

Canady, M. A., Tihova, M., Hanzlik, T. N., Johnson, J. E., and Yeager, M. Large conformational changes in the maturation of a simple RNA virus, Nudaurelia capensis ω virus (NωV). J. Mol. Biol. 2000: 299,573-584.

Filman, D. J., Wien, M. W., Cunningham, J. A., Bergelson, J. M. and Hogle, J. M. Structure determination of echovirus 1. Acta. Cryst. 1998, D54, 1261-1272.

Harrison, S. C., Skehel, J. J. and Wiley, D. C. Fields Virology, Editors B. N. Fields, D. M. Knipe, P. M. Howley, et al. Chapter 3, Virus structure. P53-98.

Johnson, J. E. and Chiu, W. Structures of virus and virus-like particles. Current Opinion in Structural Biology 2000:10,229-235.

Johnson, J. E., Lin, T., and Lomonossoff, G. Presentation of heterologus peptides on plant viruses. Genetics, Structure and Function. The annual review of phytopathology, 1997, 35:67-86.

Lamzin, V. S. and Perrakis, A. Current state of automated crystallographic data analysis. Nature Structural Biology, Structural Genomics Supplement, 2000, 978-981.

Lin, T., Porta, C., Lomonossoff, G., and Johnson, J. E. Structure-based design of peptide presentation on a viral surface: the crystal structure of a plant/animal virus chimera at 2.8 Å resolution. Fold Des. 1996, 1:179-187.

Matthews, B. W. Solvent content of protein crystals. J. Mol. Biol. 1968, 33: p491-497.

Oliveira, A. C., Gomes, A. M. O., Almeida, F. C. L., Mohana-Borges, R., Valente, A. P., Reddy, V. S., Johnson, J. E., and Silvia, J. L. Virus maturation targets the protein capsid to concerted disassembly and unfolding. J. Biol. Chem. 2000: 275, 16037-16043.

Rossman, M. G., Arnold, E., and Erickson, J. W. Structure of a human common cold virus and functional relationship to other picornavires. Nature 1985, 317: p145-153.

### EXAMPLE TWO

Construction of Dual System: Multiple Gene Expression Vectors

Dual system vectors comprising more than one transcriptional unit, wherein each transcriptional unit encodes a separate expressed gene sequence have been constructed. These vectors were constructed using PCR reactions to clone appropriate regulatory elements and genes into a single plasmid that allow for the monocistronic expression of multiple genes from the single plasmid. The parental vector from which all regulatory elements were derived in Example Two is the pTriEx plasmid available from Novagen, INc. The initial system described herein is designed to express 4 genes.

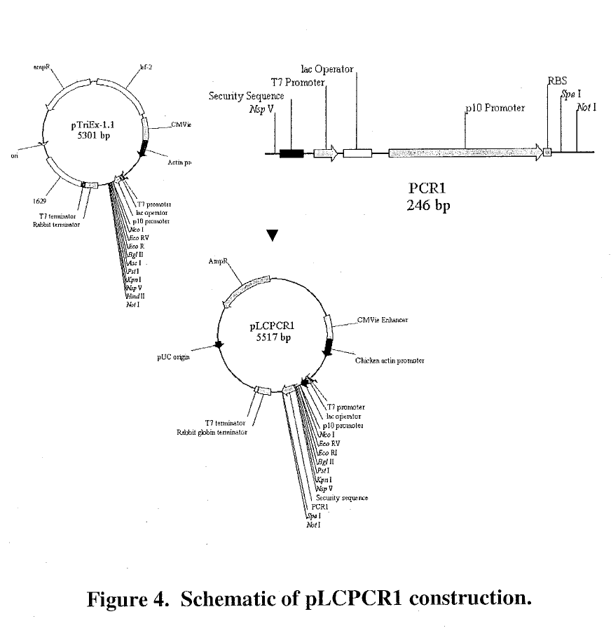

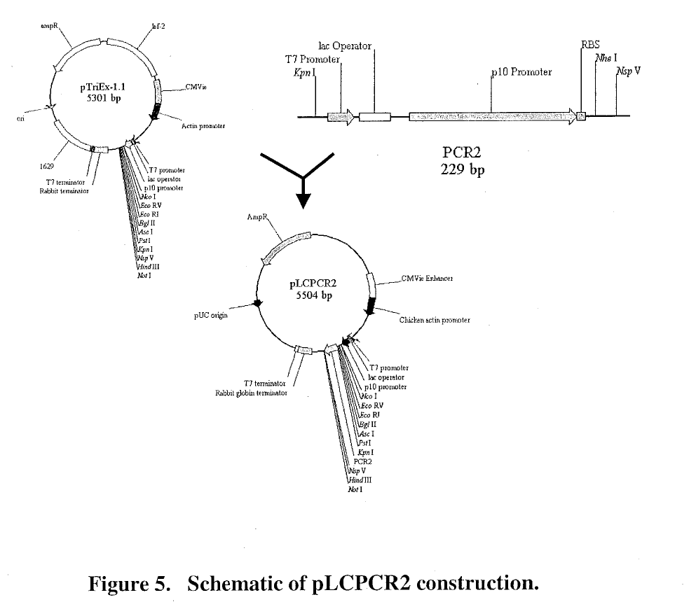

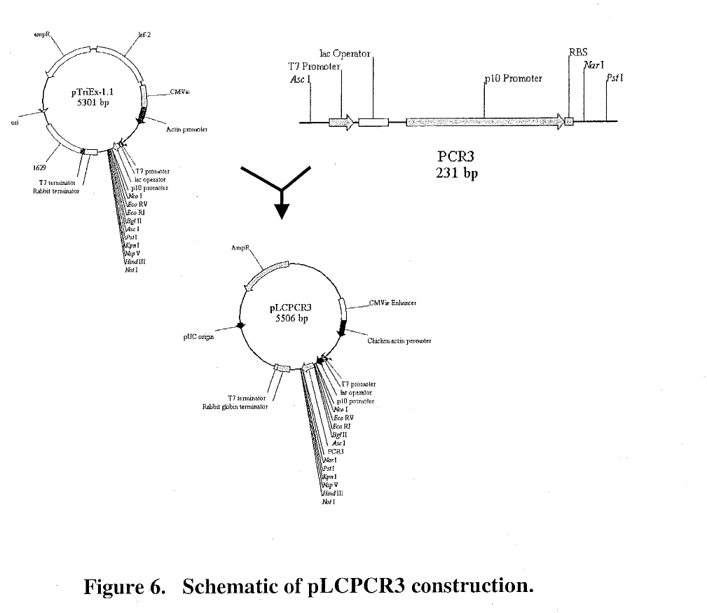

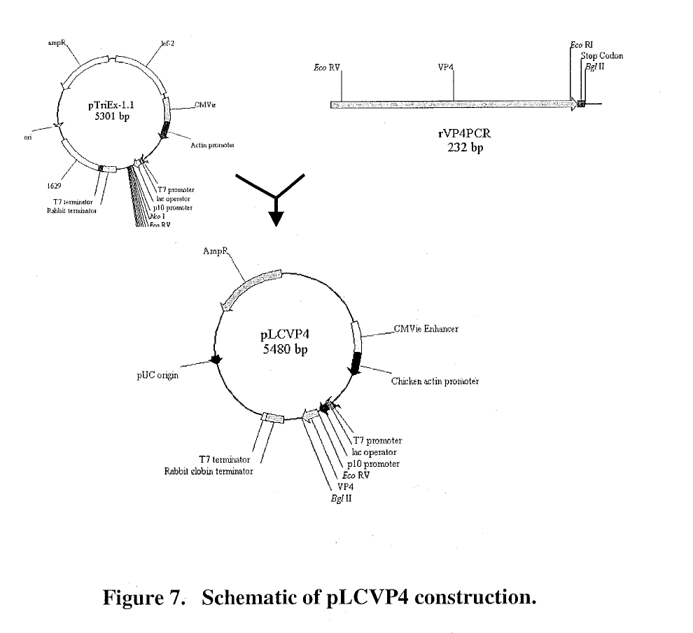

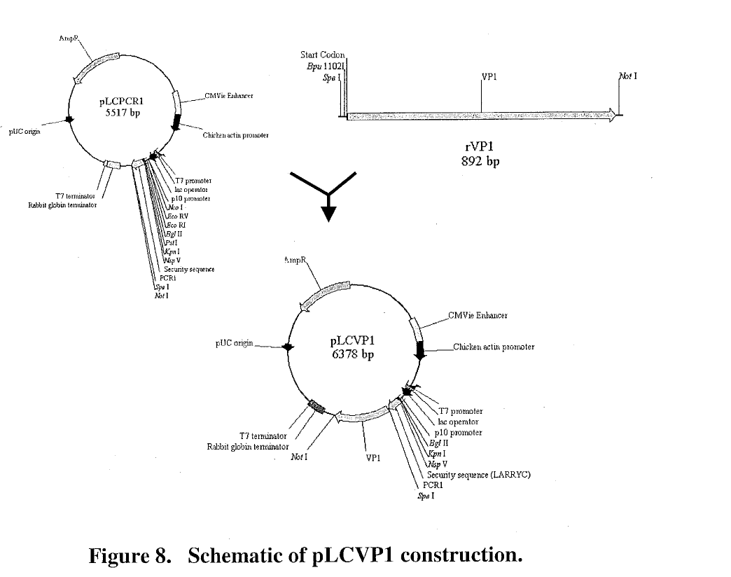

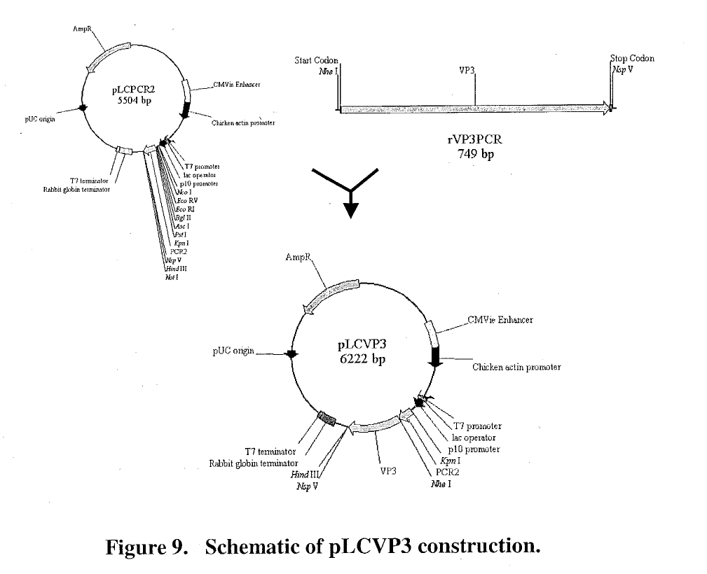

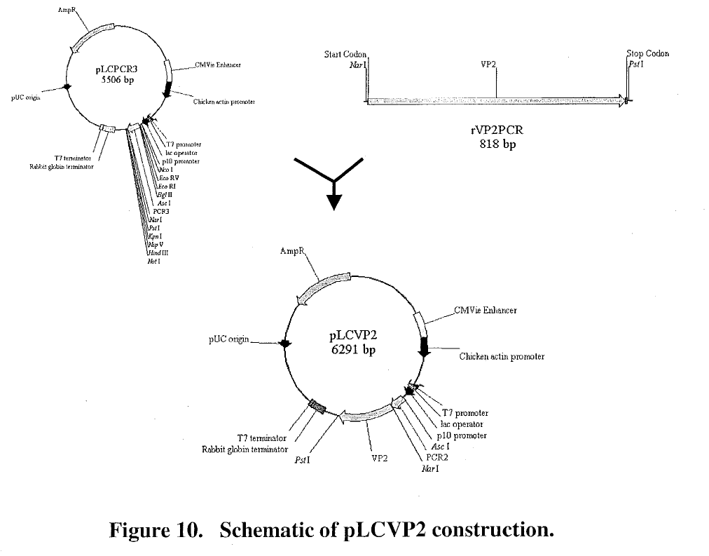

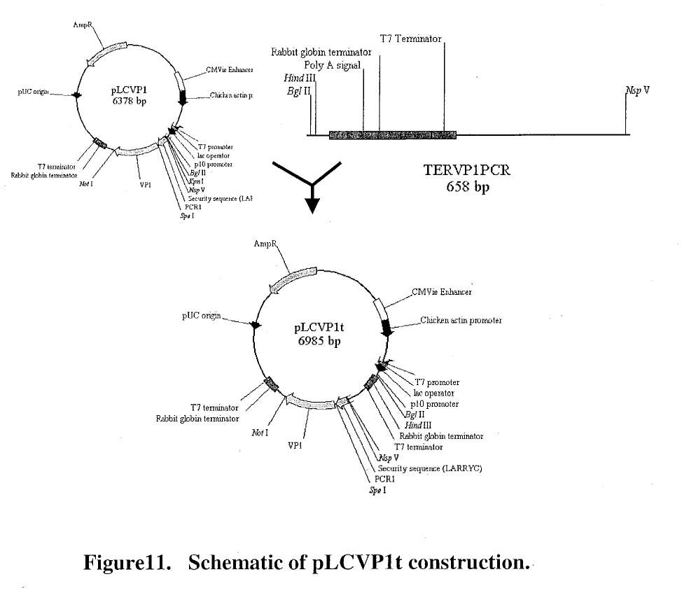

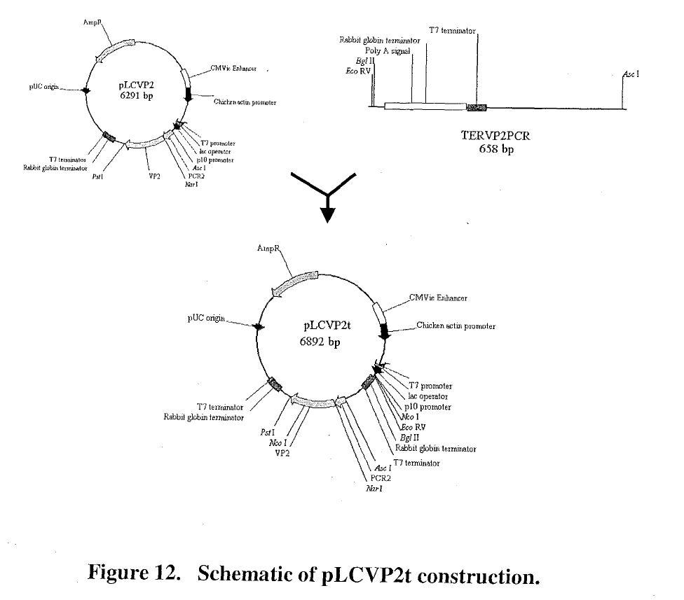

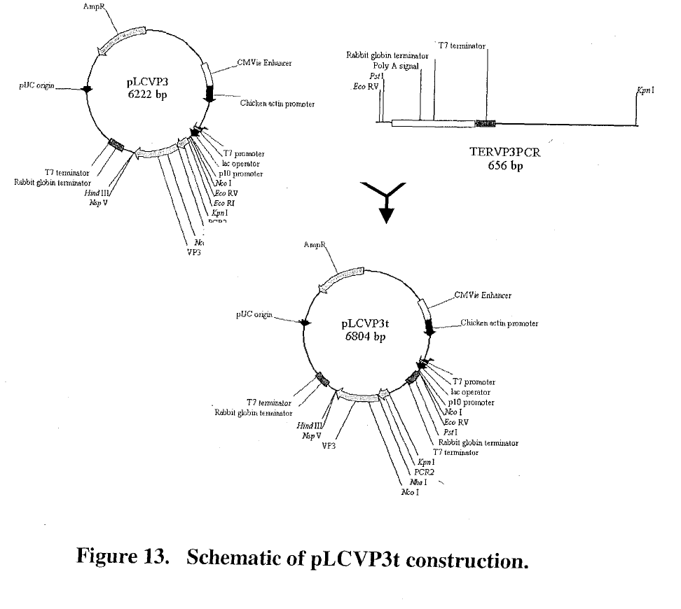

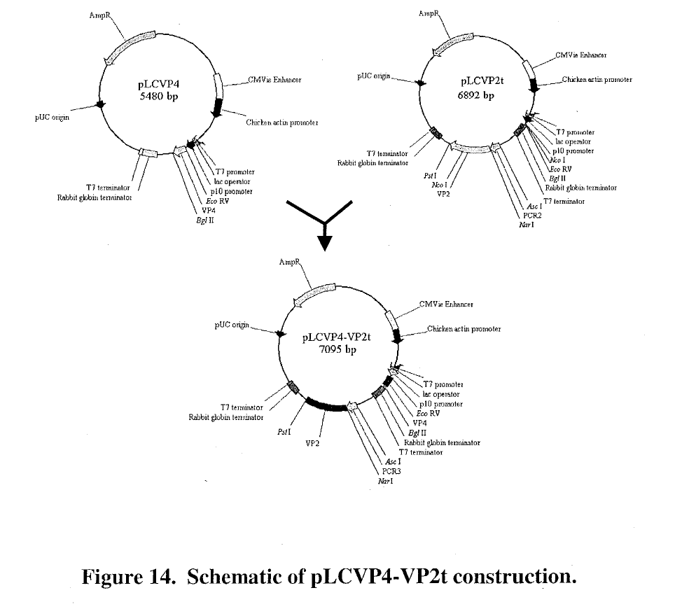

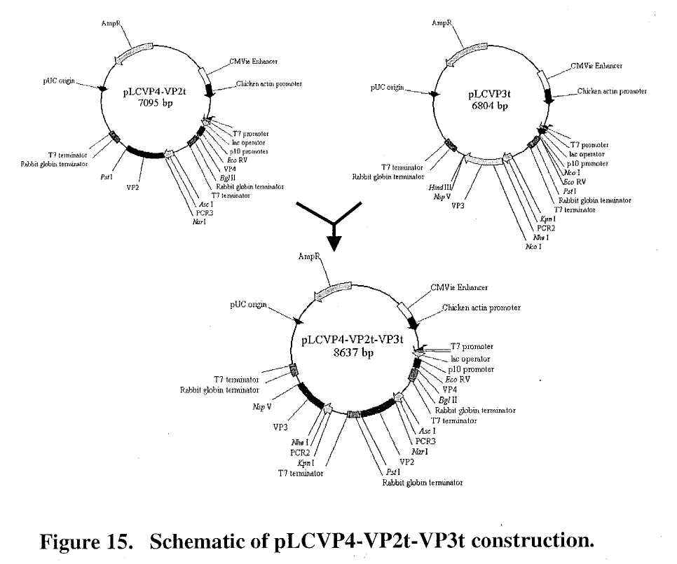

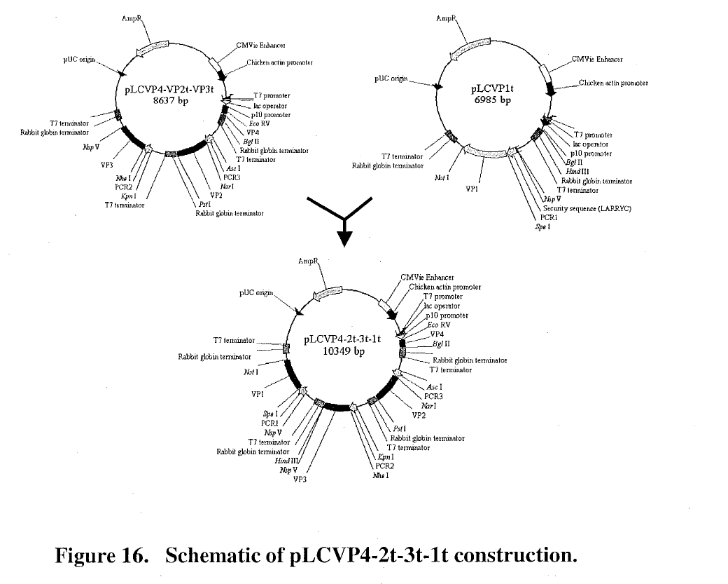

The expression of each gene is driven by a composite or hybrid promoter, T7lac/p10. This promoter is constructed from three promoter elements; two prokaryotic bacterial promoters, specifically the T7 promoter and the lac operon, and one eukaryotic promoter, specifically the p10 promoter. This promoter is also referred to herein as the tri composite promoter. Further, the presently described vector includes multiple copies of a ribosome binding site (RBS). In producing the presently described vectors, the tri composite promoter and RBS from pTriEx-1.1 was duplicated three times and then cloned back into the pTriEx-1.1 to form plasmids pLCPCR1 (FIG. 4), pLCPCR2 (FIG. 5), and pLCPCR3 (FIG. 6). Four target genes were cloned from echovirus 1 using reverse transcriptase (RT)-PCR. These gene targets were cloned into pTriEx-1.1, pLCPCR1, pLCPCR2, pLCPCR3 resulting in the construction of plasmids pLCVP1 (FIG. 8), pLCVP2 (FIG. 10), pLCVP3 (FIG. 9), and pLCVP4 (FIG. 7). DNA sequences that contain termination signals for both the prokaryotic promoter elements were cloned by PCR to form plasmids pLCVP1t (FIG. 11), pLCVP2t (FIG. 12), and pLCVP3t (FIG. 13). The regions of DNA sequence that supported the expression of each of these regions, i.e., all promoter, gene and regulatory regions, including terminator regions, were then serially cloned together to form pLCVP4-VP2t (FIG. 14), pLCVP4-VP2t-VP3t (FIG. 15), and pLCVP4-2t-VP3t-1t (FIG. 16). Vector pLCVP4-VP2t can express two genes, pLCVP4-VP2t-VP3t can express three genes, and pLCVP4-2t-3t-1t can express four genes. These genes are expressed, in this example, in the same order found in the native echovirus 1 genome. Each of the genes expressed in these examples are expressed as monocistronic mRNAs. Further details regarding the construction and properties of each of the described vectors can be seen in each of the figures in light of the knowledge of those having ordinary skill in this particular art.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

TABLE ONE
Examples of Suitable Viruses and Phage

| | | T | Space | Resolution | PDB |
|---|---|---|---|---|---|
| Virus Name | Family | Number | Group | Å | Identifier |
| Alfalfa Mosaic Virus | Bromoviridae | 1 | P63 | 4.0 | N/A |
| Bacteriophage FR | Leviviridae | 3 | C2 | 3.5 | 1frs |
| Bacteriophage G4 | Microviridae | 1 | P6322 | 3.0 | 1gff |
| Bacteriophage GA | Leviviridae | 3 | I222 | 3.4 | 1gav |
| Bacteriophage HK97 | Siphoviridae | 71 | P1211 | 3.6 | 1fh6 |
| Bacteriophage HK97 | Siphoviridae | 71 | Model* | — | 1if0 |
| ProheadII | | | | | |
| Bacteriophage MS2 | Leviviridae | 3 | R32 | 2.8 | 2ms2 |
| Bacteriophage PP7 | Leviviridae | 3 | P1 | 3.5 | 1dwn |
| Bacteriophage Qβ | Leviviridae | 3 | C2221 | 3.5 | 1qb |
| Bacteriophage ΦX174 | Microviridae | 1 | I213 | 3.5 | 2bpa |
| Bean Pod Mottle Virus | Comoviridae | P3 | P22121 | 2.8 | 1bmv |
| Black Beetle Virus | Nodaviridae | 3 | P4232 | 2.8 | 2bbv |
| Bluetongue Virus | Reoviridae | 13 | P21212 | 3.5 | 2btv |
| Bovine Enterovirus | Picornaviridae | P3 | P21 | 3.0 | 1bev |
| Carnation Mottle Virus | Tombusviridae | 3 | I23 | 3.2 | 1cmtv |
| Cowpea Chiorotic | Bromoviridae | 3 | P21212 | 3.2 | 1cwp |
| Mottle Virus | | | | | |
| Cowpea Mosaic Virus | Comovirus | P3 | I23 | 2.8 | N/A |
| Coxsackievirus B3 | Picornaviridae | P3 | P21 | 3.0 | 1cov |
| Cricket Paralysis | Picornaviridae | P3 | I222 | 2.4 | 1b35 |
| Virus 1 | | | | | |
| Cucumber Mosaic Virus | Bromoviridae | 3 | P23 | 3.2 | 1f15 |
| Densovirus | Parvoviridae | 1 | P41212 | 3.6 | 1dnv |
| Desmodium Yellow | Tymovirus | 3 | P4232 | 2.7 | 1ddl |
| Mottle Virus | | | | | |
| Echovirus 1 | Picornaviridae | 1 | P22121 | 3.55 | 1evi |
| Feline Panleukopenia | Parvoviridae | 1 | P21212 | 3.3 | 1fpv |
| | | | 1 | | |
| Flock House Virus | Nodaviridae | 3 | R3 | 3.0 | N/A |
| Foot and Mouth Disease | Piconaviridae | P3 | I222 | 3.0 | 1bbt |
| Virus | | | | | |
| Human Rhinovirus 16 at | Picornaviridae | P3 | P22121 | 2.15 | 1aym |
| high resolution | | | | | |
| Human Rhinovirus | Picornaviridae | P3 | P6322 | 3.0 | 1rla |
| HRV1A | | | | | |
| HRV Serotype 2 | Picornaviridae | P3 | I222 | 2.6 | 1fpn |
| HRV Serotype 3 | Picornaviridae | P3 | P21221 | 3.0 | 1rhi |
| HRV Serotype 14 | Picornaviridae | P3 | P213 | 3.0 | 4rhv |
| Mengo | Picornaviridae | P3 | P21212 | 3.0 | 2mev |
| Encephalomyocarditis | | | 1 | | |
| Virus | | | | | |
| Nodamura Virus | Nodaviridae | 3 | P21 | 3.3 | 1nov |
| Norwalk Virus Capsid | Caliciviridae | 3 | P42212 | 3.4 | 1ihm |
| Nudaurelia Capensis ω | Tetraviridae | 4 | P1 | 2.8 | N/A |
| Virus | | | | | |
| Pariacoto Virus | Nodaviridae | 3 | P1211 | 3.0 | 1f8v |
| Physalis Mottle Virus | Tymovirus | 3 | R3 | 3.8 | 1qjz |
| Poliovirus type 1, | Picornaviridae | P3 | P21212 | 2.9 | 2plv |
| Mahoney Strain | | | | | |
| Poliovirus type 1, Empty | Picornaviridae | P3 | P21212 | 2.88 | 1pov |
| Capsid | | | | | |
| Poliovirus type 1 at | Picornaviridae | P3 | P21212 | 2.9 | 1asj |
| −170c | | | | | |
| Poliovirus type 2 | Picornaviridae | P3 | C2221 | 2.9 | 1eah |
| Lansing | | | | | |
| Poliovirus type 3 | Picornaviridae | P3 | I222 | 2.4 | 1pvc |
| Red Clover Mottle Virus | Comoviridae | P3 | I222 | 2.4 | N/A |
| Reovirus core | Reovirus | 1 | F432 | 3.6 | 1ej6 |
| Rice Yellow Mottle | Sobemovirus | 3 | P21 | 3.0 | 1f2n |
| Virus | | | | | |
| Satellite Panicum | Statellites | 1 | P4132 | 1.9 | 1stm |
| Mosaic Virus | | | | | |
| Satellite Tobacco | Statellites | 1 | I222 | 1.8 | 1a34 |
| Mosaic Virus | | | | | |
| Satellite Tobacco | Statellites | 1 | C2 | 2.5 | 2stv |
| Necrosis Virus | | | | | |
| Sesbania Mosaic Virus | Sobemovirus | 3 | R3 | 2.9 | 1smv |
| Southern Bean Mosaic | Sobemovirus | 3 | R32 | 2.8 | 4sbv |
| Virus | | | | | |
| Simian Virus 40 (5V40) | Papovaviridae | 7d | I23 | 3.1 | 1sva |
| Murine Polyomavirus | Papovaviridae | 7d | I23 | 3.7 | 1sid |
| Theiler MEV DA | Picornaviridae | P3 | P21212 | 2.8 | 1tme |
| | | | 1 | | |
| Theiler MEV BeAn | Picornaviridac | P3 | P4322 | 3.5 | 1tmf |
| Tobacco Necrosis Virus | Necrovirus | 3 | P4232 | 2.25 | 1c8n |
| Tobacco Ringspot Virus | Nepovirus | P3 | C2 | 3.5 | La6c |
| Tomato Bushy Stunt | Tombusviridae | 3 | I23 | 2.9 | 2tbv |
| Virus | | | | | |
| Turnip Crinkle Virus | Carmovirus | 3 | I222 | 3.2 | N/A |
| Turnip Yellow Mosaic | Tymovirus | 3 | P6422 | 3.2 | 1auy |
| Virus | | | | | |

TABLE TWO
Crystallization Conditions Database

| Name | Crystallization Conditions and Results | Reference | PDB ID |
|---|---|---|---|
| Alfalfa Mosaic Virus | Empty particles of recombinant coat protein (rCP) were crystallized | Yusibov et al., J. Gen. Virol. | N/A |
| | by dialysis of a 50 μl suspension at 12-13 mg rCP/ml against 50 ml | (1996) 77, 567-573. | |
| | 0.2 M citrate buffer, pH 4.6 at 24° C. | | |
| Bacteriophage FR | Crystals grown by hanging drop vapor diffusion method with 10 μl | Bundule and Pumpens | 1frs |
| | drops containing 25 mg protein/ml and 10% saturated ammonium | J. Mol. Biol. (1993) 232, 1005- | |
| | sulfate in 50 mM MOPS (pH 7.5) 0.02% NaN₃ equilibrated against | 1006. | |
| | 35% saturated ammonium sulfate in the same buffer system. | | |
| Bacteriophage G4 | The procapsid particles were crystallized at room temperature using | McKenna et al. | 1gff |
| | the hanging drop vapor diffusion method. The reservoir solution | J. Mol. Biol. (1996) 256, | |
| | contained 2.0% (W/V) PEG 8000 and 0.2 M KCl in 50 mM bis- | | |
| | TRIS (pH 6.8) buffer, over which was suspended a hanging | | |
| | Drop of 5 μl of reservoir solution. Large amount of precipitation | | |
| | were observed forming around the growing crystals, which started | | |
| | to appear approx. two weeks after crystal trays were set up. It was | | |
| | shown, using SDS/polyacrylamide gel electrophoresis, that during | | |
| | the crystallization process the scaffolding proteins B and D | | |
| | dissociated from the procapsid particles and precipitated, leaving the | | |
| | degraded particles to crystallize. | | |
| Bacteriophage GA | The crystallization experiments were carried out in hanging drops by | Tars et al. J. Mol. Biol. (1997) 271, | 1gav |
| | the vapor diffusion technique at room temperature (20° C.). The | 759-773. | |
| | solution in the crystallization drop was prepared by mixing 10 μl of | | |
| | phage solution with 10 μl of 5% ammonium sulfate in 0.04 M TRIS- | | |
| | HCl (pH 8.0), 0.15 M NaCl and 0.02% NaN₃. The droplets were | | |
| | equilibrated against 0.9 M NaCl in 0.04 M TRIS-HCl (pH 8.0). | | |
| | Crystals with a size of 0.6 mm were obtained in three weeks. | | |
| Bacteriophage HK97 | The Head II sample at 40-70 mg/ml (4 μl) was mixed with an equal | Wikoff et al., Acta Cryst. (2000) | 1fh6 |
| | volume of precipitant: 50 mM citrate, pH 5.0, 0.85 M ammonium | D55, 763-771. | |
| | sulfate, 1.5% PEG 8000. The mixture was drawn into a capillary | | |
| | (1.0-2.0 mm diameter); mineral oil was injected at both ends to | | |
| | prevent evaporation, and the capillary ends were sealed with wax. | | |
| Bacteriophage MS2 | Crystallization experiments were performed in hanging drops by | Valegard et al. J. Mol. Biol. (1986) | 2ms2 |
| | vapor diffusion at 37° C., 19° C. and 4° C. Crystals were grown in | 190, 587-591. | |
| | 20 μl droplets applied to the inside of the lid of sterile plastic Petri | | |
| | dish. The virus solution contained 1.0% (W/V) MS2, 0.2 M sodium | | |
| | phosphate (pH 7.4) 1.5% (W/V) NaN₃. The droplets were | | |
| | equilibrated against 0.4 M sodium phosphate (pH 7.4). | | |
| Bacteriophage PP7 | NA | NA | N/A |
| Bacteriophage Qβ | Crystals grown by hanging drop vapor diffusion method at room | Valegard et al. Acta Cryst. (1994) | 1qbe |
| | temperature. The solution in the crystallization well was prepared by | D50, 105-109. | |
| | mixing 12 μl of virus solution 8 (10 mg/ml) with 8 μl of 2% PEG | | |
| | 6000 in 0.05 M TRIS/HCl pH 7.4, 0.2 M NaCl, 0.1 mM MgSO₄, | | |
| | 0.01 mM EDTA and 0.02% (W/V) NaN₃. The droplets were | | |
| | equilibrated against 0.4 M NaCl. | | |
| Bacteriophage φX174 | Crystals grown with hanging drop vapor diffusion method using PEG | Willingmann et al. J. Mol. Biol. | 2bpa |
| | 8000 as precipitant. The reservoir solution contained 90 to 93 mM | (1990) 212, 345-350. | |
| | bis-TRIS methane at pH 6.8 and 1.5 to 2.0% ((W/V) PEG 8000. | | |
| | The hanging drop contained a mixture of 5 μl of virus solution (40 | | |
| | μg of virus) and 5 μl of reservoir solution. The reservoir was filled | | |
| | with 500 μl of solution. The hanging drops were kept at room | | |
| | temperature for 1 to 2 weeks and then transferred to the cold room at | | |
| | 4° C. for another 2 or more weeks. | | |
| Bean Pod Mottle Virus | Orthorhombic crystals of BPMV were grown at 20° C. using sitting | Sehnke et al. J. Crystal Growth | 1bmv |
| | drop vapor diffusion. The reservoir solution contained BPMV 2% | (1988) 90, 222-230. | |
| | PEG 8000 (W/V) in 0.02 M sodium phosphate buffer. The virus | | |
| | solution contained middle component at 15 mg/ml in 0.1 M of | | |
| | potassium phosphate buffer pH 7.0. 25 μl of each solution were | | |
| | mixed and the mixture was equilibrated with the reservoir solution. | | |
| | Elongated tubular crystals appeared within 7-10 days. | | |
| Black Beetle Virus | Crystals grown at 20° C. using hanging drop vapor diffusion method. | Sehnke et al. J. of Crystal Growth | 2bbv |
| | A virus solution was prepared at 8 mg/ml using sodium phosphate | (1988) 90, 222-230. | |
| | buffer in a pH range of 6.9 to 7.2. The reservoir solution contained | | |
| | 0.55 M ammonium sulfate in 0.05 M sodium phosphate buffer | | |
| | adjusted to the same pH as the solution containing the virus. 5 μl of | | |
| | virus solution were mixed with 5 μl of reservoir solution and the | | |
| | mixture was equilibrated with 1 ml of the reservoir solution. The | | |
| | crystals will grow more rapidly if the reservoir and virus solution | | |
| | were initially made 1 and 0.5% (W/V) respectively in PEG 8000. | | |
| Bluetongue Virus | Crystallization trials (for BTV 1SA) were carried out by vapor | Grimes et al. Virology (1995) | 2btv |
| | diffusion (sitting drop) using microbridges supplied by Crystal | 210, 217-220. | |
| | Microsystems. The precipitant solution in the reservoir ranged from | | |
| | 11 to 16% saturated ammonium sulfate in 0.1 M TRIS-HCl buffer, | | |
| | pH 8.0. In some trials 15% ethylene glycol was also included in the | | |
| | reservoir solution. Usually 10 μl of treated cores were mixed with 5 | | |
| | μl reservoir solution. Regular crystals grow with the morphology of | | |
| | half rhombic dodecahedra, to a diameter of 0.3 mm in approx. 4 | | |
| | weeks and then more slowly to a maximum diameter of 0.8 mm. The | | |
| | largest crystals, though fewer in number, were obtained together with | | |
| | noncrystalline aggregates, when ethylene glycol was | | |
| | incorporated in the reservoir solution. | | |
| Bovine Enterovirus | Purified virus was suspended at a concentration of 10 mg/ml in 20 | Smyth et al. J. Mol. Biol. (1993) | 1bev |
| | mM TRIS.HCl (pH 7.6) containing 50 mM NaH₂PO₄ and 0.75% | 231, 930-932. | |
| | (V/V) saturated ammonium sulfate. Then the suspended virus was | | |
| | placed in 10 μl dialysis buttons, sealed with untreated Visking tubing | | |
| | and submerged in mother liquor consisting of 100 mM NaH2PO4 | | |
| | (pH 7.6) and various quantities of saturated ammonium sulfate in the | | |
| | range 20% to 35% (V/V). Crystallizations were incubated at 20° C. | | |
| | All solutions contained sodium azide at trace concentrations to | | |
| | inhibit microbiological growth during the experiments. | | |
| Canine Parvovirus (CPV) | Both CPV full and empty particles were crystallized using the | Wu et al. Acta Cryst. (1993) D49, | 2cas |
| Empty | hanging drop method in TRIS-HCl buffer at pH 7.5 containing | 572-579. | |
| | 0.75% PEG 8000 and 8 mM CaCl₂. | | |
| Carnation Mottle Virus | Crystals were obtained in 40 μl droplets of 0.1 M TRIS-HCl buffer | Morgunova et al. FEBS Letters | N/A |
| | solution containing 40-50 mg/ml of virus and 10% saturated | (1994) 338, 267-271. | |
| | ammonium sulfate. The 15 equilibrating solution consisted of 0.1 M | | |
| | TRIS-maleic (mal)/NaOH, pH 5.03 with 25% saturated ammonium | | |
| | sulfate. Either 1.7 heptandiol or PEG 300 were added to lessen the | | |
| | number of pellets. | | |
| Cowpea Chlorotic Mottle | Crystallized by the sitting drop vapor diffusion method. The | Speir et al., Virology (1993) 193, | 1cwp |
| Virus | reservoir buffer was 0.3 M disodium succinate, 0.3 M succinic acid, | 234-241. | |
| | 1 mM sodium azide, 3.7-4.0% PEG 8000, pH 3.3. Each droplet | | |
| | consisted of 5-25 μl of virus at 20-50 mg/ml in storage buffer, added | | |
| | to an equal volume of reservoir buffer. The dishes were sealed and | | |
| | allowed to equilibrate at room temperature in darkness against 15 ml | | |
| | of reservoir buffer. | | |
| Cowpea Mosaic Virus | Cubic crystals displaying rhombic dodecahedral morphology were | Lin et al. Virology (1999) | N/A |
| | obtained by vapor diffusion. The reservoir solution was 0.4 M 17 | 265, ***-***. | |
| | ammonium sulfate, 2% PEG 8000 (W/V), and 0.05 M potassium | | |
| | phosphate at pH 7.0. The virus solution was prepared at 35 mg/ml in | | |
| | 0.05 M potassium phosphate, pH 7.0. | | |
| Coxsackievirus B3 | Crystals grown at room temperature using the sitting drop vapor- | Muckelbauer, J. K., Kremer, M., | 1cov |
| | diffusion method. The sitting drop contained 10 μl of 5 mg/ml in 50 | Minor, I., Tong, L., Zlotnick, A., | |
| | mM MES buffer, pH 6.0 with 0.75 M NaCl and the well contained 1 | Johnson, J. E. and Rossmann, | |
| | ml 2 M ammonium sulfate. | M. G. Structure determination of | |
| | | coxsackievirus B3 to 3.5 A | |
| | | resolution. Acta Cryst. (1995), | |
| | | D51, 871-887. | |
| Cricket Paralysis Virus | Crystals were grown by hangingdrop vapor-diffusion at room | Tate et al. Nature Struc. Biol. | N/A |
| | temperature. Drops consisted of 1 μl of well solution plus 1 μl virus | (1999) 6, 765-774. | |
| | at a concentration of 10 mg/ml in 200 mM NaHPO₄, pH 7.2. The | | |
| | well solution was 8% (W/V) MPEG 5000, 50 mM lithium sulfate, 50 | | |
| | mM MES, pH 6.0. | | |
| Cucumber Mosaic Virus | Crystals were grown using vapor diffusion and the sitting-drop | Smith et al. J. Virol (2000) 74, | 1f15 |
| | method. The reservoir contained 2 M sodium formate, 0.1 M sodium | 7578-7586. | |
| | acetate buffer (pH 4.6), and 0.05 to 0.125% polyethylene glycol | | |
| | (PEG) 8000. To the sitting drop, 10 μl of this solution was added to | | |
| | 8 μl of the virus solution and 2 μl of a 24 mM (10 times the critical | | |
| | micelle concentration) solution of CYMAL-5 (cyclohexyl-pentyl--D- | | |
| | maltoside) was then added. The detergent improved crystal size by | | |
| | decreasing the number of nucleation sites. It did not improve | | |
| | diffraction resolution. To prepare the crystals for freezing, drops that | | |
| | did not have usable crystals were pooled and centrifuged to remove | | |
| | precipitate. This solution was then used to make 10, 20, and 30% | | |
| | solutions of PEG 400. The crystals were transferred to the increasing | | |
| | PEG solutions, with 0.5-h incubations at each step. The crystals were | | |
| | then frozen in a liquid nitrogen stream that was at 110K. | | |
| Densovirus | 10 mM TRIS pH 7.5, 1 mM CaCl₂, 1 mM MgCl₂, 0.1 M NaCl, 5% | PDB entry | 1dnv |
| | PEG 8000, (soaked in 25% glycerol for 4 hours as cryo-protectant) | | |
| Echovirus 1 | Virus was crystallized by microdialysis against 10 mM PIPES, 22 | Filman, D. J., Wien, MW., | 1ev1 |
| | 25 mM CaCl₂, 25 mM MgCl₂, 2.5% PEG 400, pH 6.0 at 4 Crystals | Cunningham, J. A., Bergelson, | |
| | grown at 20° C. | J. M. and Hogle, J. M. Structure | |
| | | determination of echovirus 1. | |
| | | Acta Cryst. (1998) D54, 1261- | |
| | | 1272. | |
| Feline Panleukopenia | Useful crystals were obtained for both full and empty particles at | Agbandje et al. | 1fpv |
| | room temperature, with PEG 8000 as precipitant. The reservoir | Proteins: Struc. Func. Gen. (1993) | |
| | solution contained 0.75% (W/V) PEG 8000 and 8 mM CaCl₂ in 10 | 16, 155-171. | |
| | mM TRIS-HCI (pH 7.5) buffer, over which was suspended a hanging | | |
| | drop of 5 μl of virus diluted by 5 μl of reservoir solution. Crystals | | |
| | grew in a period of 2 weeks or longer. | | |
| Flock House Virus | Crystallized by sitting drop vapor diffusion method. The reservoir | Fisher et al. Acta. Cryst. (1992). | 1fhv |
| | buffer was 0.01 M bis(2-hydroxyethyl)iminotris | B48, 515-520 | |
| | hydroxy-methyl)methane (bis-TRIS), 0.02 M CaCl₂, 2.8% (W/V) | | |
| | PEG 8000, pH 6.0. The drop consisted of 10 μl of FHV at 18 mg/ml | | |
| | in 0.01 M TRIS.HCl pH 7.2, plus 10-30 μl of reservoir buffer. The | | |
| | dish was sealed and allowed to equilibrate against 13 ml of reservoir | | |
| | buffer at room temperature. | | |
| Foot and Mouth Disease | Purified virus was crystallized either by dialysis in 5 to 100 μl of | Fox et al. J. Mol. Biol. (1987) 196, | 1bbt |
| Virus | ammonium sulfate in 0.1 M sodium phosphate (pH 7.6), containing a | 591-597. | |
| | trace of NaN₃ as a preservative or in vapor diffusion chambers in | | |
| | which the virus droplet had been diluted with an equal volume of the | | |
| | ammonium sulfate solution in the reservoir. All crystallizations were | | |
| | carried out at the room temperature. | | |
| Hepatitis B Virus | T = 3 and T = 4 capsids were crystallized by the vapor diffusion | Zlotnick et al. Acta Cryst. (1999) | 1qgt |
| | method. Crystals of T=4 capsids were grown from 100 mM | D55, 717-720. | |
| | NaHCO3 pH 9.5, 100 mM NaCl, 250-350 mM KCl, 9.0-9.5% | | |
| | polyethylene glycol monomethyl ether 5000 (PEG-MME) and 10% | | |
| | 2-propanol diluted 1:1 with freshly prepared capsids (10 mg/ml in 50 | | |
| | mM HEPES pH 7.5, 100 mM KCl). Crystals grew to maximum | | |
| | dimensions of 0.7 × 0.4 × 0.3 mm. Crystals of T=3 capsids grew in 2 | | |
| | weeks from 100 mM NaHCO3 pH 9.5, 100 mM NaCl, 250 mM | | |
| | LiCl, 8-8.5% PEG-MME, 10% 2-propanol. Crystals of T=3 capsids | | |
| | diffracted to approx. 8°; crystals of T=4 diffracted to 4° resolution. | | |
| Human Rhinovirus | 50 μl of 3 to 5 mg virus/ml was placed into micro-dialysis button, | Kim et al. J. Mol. Biol. (1989) | 1r1a |
| (HRV) 1A | sealed with membrane and dialyzed at 6° C. against 0.15 M | 210, 91-111. | |
| | ammonium formate adjusted to pH 7.35. Long hexagonal shaped | | |
| | crystals were obtained within 2 weeks. | | |
| HRV 2 | HRV 2 crystallized in three different morphologies using the hanging | Verdaguer et al. Acta Cryst. | 1fpn |
| | drop vapor diffusion method. Typically 2-5 μl of virus | (1999) D55, 1459-1461. | |
| | Solution (5 mg/ml) in 50 mM TRIS-HCl (pH 7.4) was mixed with an | | |
| | equal or smaller volume of reservoir solution. The cyrstals with | | |
| | prismatic morphology anddimensions up to 0.3 × 0.2 × 0.15 mm | | |
| | diffracted to high resolution (beyond 1.8°). The crystals were grown | | |
| | at room temperature and pH 7.5 using 0.4 M ammonium sulfate and | | |
| | 0.1 M sodium/potassium phosphate. | | |
| HRV 3 | The hanging drop method was used to crystallize HRV3. The | Zhao et al. Structure (1996) 4, | 1rhi |
| | reservoir solution contained 10 mM CaCl₂ and 0.75% PEG 8000 in a | 1205-1220. | |
| | 0.25 M HEPES/0.75 M NaCl/pH 7.2 buffer. The hanging drop | | |
| | contained 5 μl of 10 mg/ml virus mixed with 5 μl of reservoir | | |
| | solution. | | |
| HRV 14 | Crystals were grown at room temperature in vapor diffusion cells that | Erickson et al. Proc. natl. Acad. Sci. | 4rhv |
| | were coated with Dow Corning 4 compound to reduce nucleation and | USA (1983) 80, 931-934. | |
| | to prevent crystals from adhering to the glass surface of the wells. A | | |
| | solution of ammonium sulfate (x% saturated) containing 100 mM | | |
| | sodium phosphate buffer at pH 7.2 and 1 mM sodium azide was | | |
| | added to an equal volume of a solution containing R14 virus at y | | |
| | mg/ml (in which 2 < y < 20 mg/ml) such that the product xy was | | |
| | numerically between 5 and 10 units. The solution was put into a well | | |
| | of the diffusion chamber and equilibrated against ammonium sulfate | | |
| | at around 2.5% saturation. Crystals then grew up to 0.6 mm in length | | |
| | within a few days to a week. | | |
| HRV 16 | The hanging drop vapor diffusion method was employed in the | Oliveira et al. Structure (1993) 1, | 1aym |
| | crystallization of HRV 16. The resevoir solution (0.5 ml in volume) | 51-68. | |
| | contained PEG 8000 (0.5-1.5%) in buffer. A 5 μl drop of virus | | |
| | solution, concentrated to 8-10 mg/ml, was diluted with 5 μl of | | |
| | reservoir solution. The drop was placed on a plastic coverslip which | | |
| | was used to seal the well. Conditions for crytsallization varied with | | |
| | respect to CaCl₂ concentration present in the well solution (5-20 | | |
| | mM). A key factor in the crystallization of HRV 16 was the use of | | |
| | NaCl in the buffer. | | |
| Mengo | An orthorhombic crystals were prepared by hanging drop vapor | Luo et al., Science (1987) 235, | 2mev |
| Encephalomyocarditis | diffusion method with 2.8% PEG 8000 in 0.1 M phosphate buffer at | 182-191. | |
| Virus | pH 7.4 in the reservoir with an initial virus concentration of 5 mg/ml | | |
| | and 1.4% PEG 8000 in the same buffer in the hanging drop. The | | |
| | crystals grew in 1-2 days at room temperature to a maximum | | |
| | dimension of 0.8 mm. | | |
| Murine Minute Virus | Crystals were grown using hanging drop vapor diffusion method with | Llamas-Saiz et al. Acta Cryst. | 1mvm |
| | conditions similar to those used for CPV. The reservoir solution | (1997) D53, 93-102. | |
| | contained 0.75% (W/V) PEG 8000 and 8 mM CaCl₂.2H2O in 10 | | |
| | mM hanging drop produced by mixing 5 ml of virus solution (10 | | |
| | mg/ml) in 10 mM TRIS-HCl at pH 7.5 with 5 μl of reservoir | | |
| | solution. Crystals grew to a maximum dimension of 0.4 mm in about | | |
| | 4 to 8 weeks. | | |
| Nodamura Virus | 10-15 μl of 7 mg/ml of virus in phosphate buffer mixed with one | PDB entry 1nov | 1nov |
| | volume of citrate buffer and equilibrated verses 20 ml of citrate | | |
| | buffer (0.24-0.28 M sodium citrate, pH adjusted to 6.0 with acetic | | |
| | acid, or 0.24 M potassium citrate pH 6.0, both with 0.1% beta-octyl | | |
| | glucopyranoside). Crystals grown from vapor diffusion using sitting | | |
| | drop method. | | |
| Norwalk Virus | Crystals of the rNV particles suitable for x-ray structure | Prasad et al., Science (1999) 286, | 1ihm |
| | determination were grown by the hanging drop method with 0.5 M | 287-290. | |
| | ammonium phosphate (pH 4.8) as the precipitant. | | |
| Nudaurelia Capensis ω | The virus crystallized using sitting drop method of vapor diffusion. | Cavarelli et al. Acta Cryst. | N/A |
| Virus | The reservoir solution was prepared using 0.075 M | (1991) B47, 23-29. | |
| | Morpholinopropanesulfonic acid (MOPS) buffer at pH 7.0 with PEG | | |
| | 8000 at 2% CaCl₂ at 0.25 M and NaN₃ at 0.00 1 M. The virus | | |
| | Solution was at 8-10 mg/ml in 0.07 M sodium acetate buffer at pH | | |
| | 5.0. The crystallization drops consisted of 10 μl of the virus solution | | |
| | mixed with 40 μl of the reservoir solution. The mixture was allowed | | |
| | to reach vapor equilibrium with the reservoir solution (20 ml). | | |
| | Tabular shaped crystals appeared in 2-4 weeks. | | |
| Physalis Mottle Virus | ? | Krishna et al., J. Mol. Biol. (1999) | 1qjz |
| | | 289, 919-934. | |
| Pariacoto Virus | PaV was crystallized by the hanging drop vapor diffusion method at | Tang et al. Nature Struc. Biol. | 1f8v |
| | room temperature. The reservoir was 1 ml of 75 mM Li₂SO₄, 5 mM | (2001) 8, 77-83. | |
| | CaCl₂, and 4% (W/V) PEG 8000 in 50 mM Tris-HCl buffer, pH 7.5. | | |
| | The droplet was a mixture of 1 μl reservoir solution and 1 μl virus | | |
| | sample at a virus concentration of aprox. 20 mg ml-1 in 50 mM Tris- | | |
| | HCl buffer, pH 7.5. Crystals appeared within 4-5 days. | | |
| Poliovirus Empty | ? | | 1pov |
| 1 | Crystals of empty capsids were grown by dialyzing 5-15 μl samples | Basavappa, R. Syed, R., Flore, O., | 2plv |
| | of empty capsid (approx. 15 mg/ml) initially in 0.8 M NaCl, PMC7 | Icenogle, J. P., Filman, D. J., and | |
| | 10 mM PIPES, 5 mM MgCl₂ at 4° C. | Hogle, J. M. Role and mechanism | |
| | | of the maturation cleavage of | |
| | | VPO in poliovirus assembly: | |
| | | Structure of the empty capsid | |
| | | assembly intermediate at 2.9 A | |
| | | resolution. Protein Science | |
| | | (1994), 3: 1651-1669. | |
| 2 Lansing | Crystals were grown at room temperature using a modified version of | Lentz et al. Structure (1997) 5, | 1eah |
| | the hanging drop vapor diffusion method. The reservoir | 961-978. | |
| | Solution (0.5 ml total volume) contained varying amounts of PEG | | |
| | 8000 (0.9-1.4%) and lithium sulfate (100-250 mM). The virus | | |
| | Sample, 2-3 μl of a 5 mg/ml solution, was placed on a plastic | | |
| | coverslip and mixed with an equal volume of the reservoir solution. | | |
| | The well was sealed with the Coverslip using vacuum grease except | | |
| | for a small leak that was left between the coverslip and the well. | | |
| | After 2-4 days, crystals approx. 0.1 mm × 0.2 mm × 0.1 mm | | |
| | Began to appear, at which time the leak was sealed with vacuum | | |
| | grease and the crystals were allowed to grow to their maximum size | | |
| | of 0.2 mm × 0.35 mm × 0.2 mm. Without the leak, the crystallization | | |
| | drops would either form precipitate or remain clear for months. The | | |
| | leak left between coverslip and the well was a key factor in the | | |
| | production of crystals suitable for X-ray diffraction analysis. | | |
| 3 | ? | Reference | 1pvc |
| Red Clover Mottle Virus | Elongated RCMV crystals were produced by the sitting drop vapor | Lin et al., J. Virol., (2000) 74, 493- | N/A |
| | diffusion method. The starting solution contained 10 mg/ml RCMV | 504. | |
| | in 10 mM sodium phosphate, pH 7.0. The reservoir solution | | |
| | contained 50 mM potassium phosphate, pH 7.0, 1.8% PEG 8000. 0.3 | | |
| | M ammonium sulfate 2 mM EDTA and 1 mM sodium azide. Equal | | |
| | volumes of the virus and reservoir solution were mixed with the | | |
| | reservoir solution at room temperature. The crystals grew to 0.5 to 1 | | |
| | mm in all dimensions after 5 to 7 days. | | |
| Rice Yellow Mottle Virus | The crystallization was carried out by vapor diffusion and the | Qu et al., (2000) in press | 1f2n |
| | reservoir solution was 50 mM sodium citrate, pH 3.0, 200 mM | | |
| | lithium sulfate, and 3.6% (W/V) PEG 8000. The virus solution was | | |
| | concentrated to 36 mg/ml. | | |
| Satellite Panicum Mosaic | Cubic crystals grown by vapor diffusion methods using glass | Day et al. J. Mol. Biol. (1994) | 1stm |
| Virus | depression plates in plastic sandwich boxes at 4° C. over a | 238, 849-851. | |
| | period of about one month. The reservoir solution was 37% saturated | | |
| | ammonium sulfate in water. The droplets were composed of 10 μl of | | |
| | a 10 mg/ml virus solution (buffered with 20 mM potassium | | |
| | phosphate) plus 10 μl of the reservoir. | | |
| Satellite Tobacco Mosaic | Protein was four times recrystallized from bulk solution by addition | PDB entry 1a34 | 1a34 |
| Virus | AF ammonium sulfate to 15% saturation. Space crystals were grown | | |
| | by liquid-liquid diffusion in a microgravity environment over 12 | | |
| | days aboard IML-I mission of the US space shuttle. | | |
| Satellite Tobacco | Crystals grown from solutions containing 10-12 g of virus/l (or 7-8 g | Lilijas et al., J. Mol. Biol. (1982) | 2stv |
| Necrosis Virus | of virus/l and 0.4% (W/V) PEG 6000) in 1 mM Mg (2+), 50 mM 93- | 159, | |
| | 108. Sodium phosphate pH 6.5. | | |
| Sesbania Mosaic Virusin | The purified virus was crystallized by vapor diffusion in depression | Subramanya et al. | 1smv |
| | slides. Best crystals were obtained by precipitating the virus (30 | J. Mol. Biol. (1993) 229, 20-25. | |
| | mg/ml 0.1 M sodium acetate (pH 5.6)) with 15% to 20% saturated | | |
| | ammonium sulfate in the inner well and 30% saturated in the outer | | |
| | well. Addition of divalent salts had pronounced effect on crystal | | |
| | growth. | | |
| Southern Bean Mosaic | The virus was crystallized in vials from 0.95 M ammonium sulfate | Johnson et al. J. Ultrastruc. Res. | 4sbv |
| Virus | with an initial virus concentration of 20 mg/ml. | (1974) 46, 441-451. | |
| Simian Virus 40 | Crystals were grown at 25° C. (by hanging drop technique) from a | Lattman et al. Science (1980) | 1sva |
| | solution containing approx. half-saturated ammonium sulfate | 208, 1048-1050. | |
| | buffered with either TRIS (hydroxymethyl) aminomethane or | | |
| | ammonia to pH 7.0 to 7.5, 10 mM Mg (2+) and 0.5 mM Ca (2+). The | | |
| | concentration of virus was 5 to 10 mg/ml. Morphologically the | | |
| | crystals were cubes. | | |
| Murine Polyomavirus | Crystals were grown from sodium sulfate using hanging drop method | Stehle and Harrison, Structure | 1sid |
| | and salanized coverslips. The 2 μl drops contained 6-8 mg/ml virus, | (1996) 4, 183-194. | |
| | 10 mM HEPES pH 7.5, 0.25-0.3 M sodium sulfate and 2.5-5.0% | | |
| | (V/V) glycerol; the reservoir contained 0.55-0.6 M sodium sulfate, | | |
| | 10 mM HEPES pH 7.5 and 5-10% glycerol. Harvest buffer contained | | |
| | 0.65 M sodium sulfate, 50 mM HEPES pH 7.5 and | | |
| | 10% glycerol. For oligosaccharide complex formation, the crystals | | |
| | were soaked in harvest buffer 24 h prior to data collection. | | |
| Theiler MEV BeAn | Crystals grown by hanging drop vapor diffusion method with PEG | Luo et al. | 1tmf |
| | 3350 in 0.02 M boric acid buffer (pH 8.5). | Proc. Natl. Acad. Sci. USA (1992) | |
| | | 89, 2409-2413. | |
| Theiler Murine | Concentrated samples of virus (10 mg/ml) were crystallized at 4° C. | Grant et al. | 1tme |
| Encephalo-Myelitis Virus | by microdialysis verses progressively lower concentrations of NaCl | Proc. Natl. Acad. Sci. USA (1992) | |
| DA | in 10 mM Na PIPES buffer (pH 7.0-7.3). | 89, 2061-2065. | |
| Tobacco Necrosis Virus | Crystals grown by dialysis method using microdialysis cells by both | Fukuyama et al. J. Mol. Biol. | 1c8n |
| | lowering pH and increasing salt concentration. Virus solution was | (1987) 196, 961-962. | |
| | dialyzed against 0.4 M sodium | | |
| | Phosphate buffer with the pH adjusted to 6.0. Sometimes thin plate | | |
| | like crystals were produced with the dodecahedral crystals in the | | |
| | same dialysis cells. The thin plate like crystals were dissolved by | | |
| | dialyzing against 10 mM sodium phosphate buffer (pH 7.0). When | | |
| | the cells were transferred to the crystallization buffer, dodecahedral | | |
| | crystals were usually produced. | | |
| Tobacco Ringspot Virus | Virus was crystallized using hanging drop setting from reservoir | PDB entry 1a6c | 1a6c |
| | buffer containing 2-3% (W/V) PEG 3350, 1 mM sodium azide and | | |
| | 0.125 M potassium phosphate, pH 6.5. | | |
| Tomato Bushy Stunt | The virus was crystallized by adding saturated ammonium sulfate to | Harrison and Jack, J. Mol. Biol. | 2tbv |
| Virus | the virus (approx. 30 mg/ml in water) until the solution just remained | (1975) 97, 173-191. | |
| | turbid. The final concentration of ammonium sulfate at this endpoint | | |
| | was approx. 0.5 M but varied from preparation to preparation. The | | |
| | solution was distributed into stoppered vials and stored at 4° C. At | | |
| | this temperature the turbidity vanished and single crystals grew | | |
| | after a period of weeks or months. Seeding accelerated the process, | | |
| | but several months were necessary to obtain large crystals (0.3 to 0.5 | | |
| | mm). | | |
| Turnip Crinkle Virus | Well-ordered crystals could be grown only as the methyl mercury | Hogle et al. J. Mol. Biol. (1986) | N/A |
| | adduct; the corresponding native crystals have a complex packing | 191, 625-638. | |
| | disorder. The methyl mercury adduct was obtained by bringing | | |
| | stock solution of virus (3.5% TCV (W/V) in 0.01% NaN₃) to 6 | | |
| | equivalent methyl/protein subunit by addition of 15 mM methyl | | |
| | mercury nitrate and incubating for 1 hr. Crystallization was then | | |
| | initiated by addition of an approx. equal volume of saturated sodium | | |
| | citrate (pH 7.0) and allowed to proceed undisturbed for 2 to 4 | | |
| | months. The optimum concentration of sodium citrate required to | | |
| | produce large crystals varied from experiment to experiment, but was | | |
| | generally in the range of 42 to 46% saturated. | | |
| Turnip Yellow Mosaic | Crystals grown using hanging drop vapor diffusion technique. The | Canady et al. (1995) Proteins: | 1auy |
| Virus | reservoir solution contained and 1.17 M ammonium phosphate and | Struc. Func. Gen. 21, 78-81. | |
| | 100 mM MES buffer with a final pH of 3.7-5.5. 5 μl of virus solution | | |
| | (16 mg/ml) 5 μl of reservoir solution composed of the micro-drops | | |
| | yielded large crystals at 25° C. | | |

(adapted from http://mmtsb.scripps.edu/viper.viper.html)

That which is claimed is:

1. A chimeric capsid protein comprising:
a first polypeptide sequence and a second polypeptide sequence, wherein;
(a) the first polypeptide sequence consists of native capsid protein amino acid sequence;
(b) the second polypeptide sequence consists of a heterologous non-capsid amino acid sequence; and
(c) the second polypeptide sequence is displayed on the surface of the chimeric capsid protein which lies on the inner surface of a phage or viral capsid formed from the capsid protein.

2. The chimeric capsid protein of claim 1, wherein the first polypeptide sequence is derived from a phage.

3. The chimeric capsid protein of claim 2, wherein the phage is selected from a list consisting of bacteriophage FR, bacteriophage G4, bacteriophage GA, bacteriophage HK97, bacteriophage HK97 proheadII, bacteriophage MS2, bacteriophage PP7, bacteriophage Qβ and bacteriophage ΦX174.

4. The chimeric capsid protein of claim 2, wherein the phage is an unenveloped phage.

5. The chimeric capsid protein of claim 2, wherein the phage is an isometric phage.

6. The chimeric capsid protein of claim 1, wherein the first polypeptide sequence is derived from a virus.

7. The chimeric capsid protein of claim 6, wherein the virus is selected from a list consisting of echovirus 1, hepatitis B virus, alfalfa mosaic virus, bean pod mottle virus, black beetle virus, bluetongue virus, bovine enterovirus, carnation mottle virus, cowpea chlorotic mottle virus, cowpea mosaic virus, coxsackievirus B3, cricket paralysis virus, cucumber mosaic virus, densovirus, desmodium yellow mottle virus, feline panleukopenia virus, flock house virus, foot and mouth disease virus, human rhinovirus 16, human rhinovirus HRV1A, human rhinovirus serotype 2, human rhinovirus serotype 3, human rhinovirus serotype 14, meno encephalomyocarditis virus, nodamura virus, Norwalk virus, nudaurelia capensis ω virus, pariacoto virus, physalis mottle virus, poliovirus type 1, poliovirus type 2 Lansing, poliovirus type 3, red clover mottle virus, reo virus, rice yellow mottle virus, satellite panicum mosaic virus, satellite tobacco mosaic virus, satellite tobacco necrosis virus, sesbania mosaic virus, southern bean mosaic virus, simian virus 40, murine polyomavirus, Theiler MEV DA, Theiler MEV BeAn, tobacco necrosis virus, tobacco ringspot virus, tomato bushy stunt virus, turnip crinkle virus and turnip yellow mosaic virus.

8. The chimeric capsid protein of claim 6, wherein the virus is an unenveloped virus.

9. The chimeric capsid protein of claim 6, wherein the virus is an isometric virus.

10. The chimeric capsid protein of claim 1, wherein the second polypeptide sequence is derived from a species different from the species from which the first polypeptide sequence is derived.

11. The chimeric capsid protein of claim 10, wherein the second polypeptide sequence comprises rhodopsin and portions or functional derivatives thereof.

12. The chimeric capsid protein of claim 10, wherein the second polypeptide sequence comprises cytochrome p450 and portions or functional derivatives thereof.

13. The chimeric capsid protein of claim 10, wherein the chimeric capsid protein comprises a detectable protein label.

14. The chimeric capsid protein of 13, wherein the detectable protein label is a green fluorescent protein or functional portions thereof.

15. The chimeric capsid protein of 13, wherein the detectable protein label is an enzymic label in which a substrate or product of a reaction catalyzed by the enzymic label is a detectable reporter agent.

16. The chimeric capsid protein of 15, wherein the enzymic label is horseradish peroxidase or functional portions thereof.

17. The chimeric capsid protein of claim 10, wherein the second polypeptide sequence retains biological activity when incorporated in the chimeric capsid protein.

18. The chimeric capsid protein of claim 17, wherein the second polypeptide sequence binds to a nucleic acid.

19. The chimeric capsid protein of claim 18, wherein the second polypeptide sequence binds to specified nucleic acid sequences.

20. The chimeric capsid protein of claim 17, wherein the nucleic acid is DNA.

21. The chimeric capsid protein of claim 17, wherein the second polypeptide sequence binds to nucleic acids with specified structures.

22. The chimeric capsid protein of claim 21, wherein the specified structure is double-stranded.

23. The chimeric capsid protein of claim 21, wherein the specified structure is single-stranded.

24. The chimeric capsid protein of claim 21, wherein the specified structure is that of a regulatory element.

25. The chimeric capsid protein of claim 17, wherein the second polypeptide binds to an antigen.

26. The chimeric capsid protein of claim 25, wherein the second polypeptide is an antibody.

27. The chimeric capsid protein of claim 17, wherein the second polypeptide is a protease.

28. The chimeric capsid protein of claim 17, wherein the second polypeptide comprises amino acid sequence derived from a necessary protein whose function is required to prevent, cure or ameliorate a diseased state.

29. The chimeric capsid protein of claim 28, wherein the necessary protein is not present at adequate levels or is defective in function in a subject suffering from a diseased state.

30. The chimeric capsid protein of claim 29, wherein the necessary protein is selected from the group consisting of alpha glucosidase, glucocerebrosidase, glucose-6-phosphatase, atp7b protein and uridine diphosphate glycosyl transferase.

31. The chimeric capsid protein of claim 28, wherein the presence of the necessary protein is not required at the levels required to prevent, cure or ameliorate a diseased state in a subject not suffering from a diseased state or a predisposition towards a diseased state.

32. The chimeric capsid protein of claim 17, wherein the second polypeptide is a nuclease.

33. The chimeric capsid protein of claim 32, wherein the nuclease is an endonuclease.

34. The chimeric capsid protein of claim 32, wherein the nuclease is an exonuclease.

35. The chimeric capsid protein of claim 32, wherein the nuclease is a deoxyribonuclease.

36. The chimeric capsid protein of claim 32, wherein the nuclease is a ribonuclease.

37. The chimeric capsid protein of claim 17, wherein the second polypeptide is cytotoxic.

38. The chimeric capsid protein of claim 37, wherein the second polypeptide is greater than 5 amino acid residues in length.

39. The chimeric capsid protein of claim 38, wherein the second polypeptide is greater than 25 amino acid residues in length.

40. The chimeric capsid protein of claim 39, wherein the second polypeptide comprises the catalytic domain of diphtheria toxin.

41. The chimeric capsid protein of claim 17, wherein the chimeric capsid protein is cytotoxic.

42. A capsid comprising the chimeric capsid protein of claim 1.

43. The capsid of claim 42, wherein the only capsid protein is the chimeric capsid protein of claim 1.

44. The capsid of claim 42, wherein the capsid comprises both the chimeric capsid protein of claim 1 and further capsid proteins.

45. The capsid of claim 44, wherein the further capsid proteins including a protein from which the first polypeptide sequence was derived.

46. The capsid of claim 42, wherein the capsid is unenveloped.

47. The capsid of claim 42, wherein the capsid is isometric.

48. The capsid of claim 31, wherein the capsid forms without packaging nucleic acid.

49. The capsid of claim 48, wherein a nucleic acid encoding the capsid proteins is physically occluded from the interior of the capsid.

50. The capsid of claim 48, wherein a nucleic acid encoding the capsid proteins is not physically occluded from the interior of the capsid.

51. A repetitive ordered structure comprising the capsids of claim 42.

52. The ordered structure of claim 51, wherein the capsids form a two-dimensional array.

53. The ordered structure of claim 52, wherein the capsids are immobilized on a solid support.

54. The ordered structure of claim 52, wherein the capsids are immobilized on a membrane, a lipid monolayer or a lipid bilayer.

55. The ordered structure of claim 51, wherein the capsids form a three-dimensional array.

56. The ordered structure of claim 55, wherein the capsids are immobilized on a solid support.

57. The ordered structure of claim 55, wherein the capsids are immobilized on a membrane, a lipid monolayer or a lipid bilayer.

58. An isolated nucleic acid comprising a transcriptional unit encoding the chimeric capsid protein of claim 1, wherein the transcriptional unit directs the synthesis of the chimeric capsid protein.

59. The nucleic acid of claim 58, wherein the nucleic acid directs the synthesis of the chimeric capsid protein in vitro, in isolated cells, in cell culture, in tissues, in organs or in organisms.

60. The nucleic acid of claim 58, wherein the nucleic acid is RNA.

61. The nucleic acid of claim 58, wherein the nucleic acid is DNA.

62. The nucleic acid of claim 61, wherein the nucleic acid is a phagemid.

63. The nucleic acid of claim 58, wherein a first region of nucleic acid sequence at the 5′ end of the nucleic acid sequence encoding heterologous amino acid sequence specifies a first restriction endonuclease cleavage site and a second region of nucleic acid sequence at the 3′ end of the nucleic acid sequence encoding heterologous amino acid sequence specifies a second restriction endonuclease cleavage site.

64. The nucleic acid of claim 63, wherein the first and second restriction endonuclease cleavage sites are for different restriction endonucleases.

65. The nucleic acid of claim 63, wherein the first and second restriction endonuclease cleavage sites are for the same restriction endonuclease.

66. A process for determining the structure of a polypeptide, comprising the steps:
(a) generating an isolated nucleic acid vector comprising a transcriptional unit encoding the chimeric capsid protein of claim 1, wherein the transcriptional unit directs the synthesis of the chimeric capsid protein;
(b) expressing the chimeric capsid protein encoded by the nucleic acid vector of step (a);
(c) forming capsids comprising the chimeric capsid protein of step (b);
(d) forming repetitive ordered arrays of the capsids of step (c);
(e) obtaining x-ray diffraction patterns of the repetitive ordered arrays of step (d); and
(f) determining an atomic level or near-atomic level structure of the capsids, or a portion thereof, wherein the structure obtained comprises the structure of the polypeptide.

67. The process of claim 66, wherein the capsids formed in step c) comprise the chimeric capsid protein of step (b) and wild-type capsid protein.

68. The process of claim 66, wherein the repetitive ordered arrays of the capsids of step (c) are crystals.

69. The process of claim 66, wherein step (f) comprises generating an electron density difference map between a crystal of fully wild-type capsid proteins and a crystal comprising chimeric capsid proteins.

70. The process of claim 69, wherein step (f) comprises use of a structure of the heterologous non-capsid amino acid sequence as a search model to determine the structure of the chimeric capsid proteins.

71. The process of claim 69, wherein step (f) comprises use of a structure of a wild-type capsid protein as a search model to determine the structure of the chimeric capsid proteins.

72. A method of characterizing the chimeric capsid proteins, comprising:
crystallizing capsids formed of the chimeric capsid proteins of claim 1 and analyzing the crystallized capsids.

73. The method of claim 72, wherein the crystallization occurs in hanging drops using a vapor diffusion method.

74. The method of claim 72, wherein the crystallization occurs in volumes of solution whose composition is altered by microdialysis.

75. The method of claim 72, wherein the analyzing is by diffraction of electromagnetic radiation or particles.

76. The method of claim 75, wherein the electromagnetic radiation is x-ray radiation.

77. The method of claim 75, wherein the particles are neutrons.

78. A method of identifying ligands of the chimeric capsid protein, comprising:
(a) contacting potential ligands of the chimeric capsid protein with the chimeric capsid protein of claim 1 under conditions whereby a ligand/protein complex can form; and
(b) detecting ligand/protein complex formation, thereby determining that the potential ligand is bound by the chimeric capsid protein.

79. A method of characterizing ligands of a chimeric capsid protein, comprising:
(a) contacting ligands of the chimeric capsid protein with the chimeric capsid protein of claim 1 thereby forming a ligand/protein complex;
(b) forming capsids of the ligand/protein complex; and
(c) analyzing the crystallized capsids.


SEQUENCE LISTING (2 sequences)

SEQ ID NO: 1 - DNA, 13 bp, Artificial Sequence
  Description of Artificial Sequence; NOTE=Synthetic construct
gccgccrccaugg

SEQ ID NO: 2 - DNA, 39 bp, Artificial Sequence
  Description of Artificial Sequence; NOTE=Synthetic construct
taaggatccttaacacaggaggcagaccatggtaccggt